(19) United States
(12) Patent Application Publication
(10) Pub. No.: US 2019/0054024 A1
(43) Pub. Date: Feb. 21, 2019

(54) Solid Dosage Forms

(71) Applicants: HUMANWELL PHARMACEUTICAL US, Inc., Ballwin, MO (US); YICHANG HUMANWELL PHARMACEUTICAL CO., LTD., Hubei (CN)

(72) Inventors: Jun Yang, Ballwin, MO (US); Yunqi Wu, Ballwin, MO (US); Dunxi Chen, Hubei (CN); Cuiying Zhang, Hubei (CN); Yuping Yang, Hubei (CN); Longmei Qu, Hubei (CN); Lie Li, Hubei (CN); Jie Li, Hubei (CN); Wentao Du, Hubei (CN); Yigang Fu, Hubei (CN)

(73) Assignees: HUMANWELL PHARMACEUTICAL US, Inc., Ballwin, MO (US); YICHANG HUMANWELL PHARMACEUTICAL CO., LTD., Hubei, MO (CN)

(21) Appl. No.: 15/678,923

(22) Filed: Aug. 16, 2017

(51) Int. Cl. A61K 9/16; A61K 9/20; A61K 9/48; A61K 9/10; A61K 47/32; A61K 47/38

(52) U.S. Cl. CPC A61K 9/1676; A61K 9/1635; A61K 9/1652; A61K 47/38; A61K 9/4808; A61K 9/10; A61K 47/32; A61K 9/2077

(57) ABSTRACT

A solid pharmaceutical dosage form having drug-loaded particles and methods of making.

### FIELD OF THE INVENTION

The present invention relates to solid dosage forms employing drug-loaded particles. The solid dosage forms may exhibit immediate release or modified release of the drug from the solid dosage form upon administration to a patient or when subjected to in vitro testing. The solid dosage forms may also exhibit abuse-deterrent features.

### BACKGROUND OF THE INVENTION

Solid dosage forms for oral administration of drugs are well known in the art. For example, tablets, capsules, granules, powders and pellets have long been used in the pharmaceutical arts for the administration of drugs to patients in need. Descriptions of these well recognized solid dosage forms and methods for preparing them can be found in various patents and reference materials and typically involve the combination of the drug with one or more pharmaceutical excipients. For example, the drug may be mixed or blended with one or more pharmaceutical excipients and the mixture filled into capsule shells or compressed into a tablet, sometimes referred to as direct compression. Alternatively, the drug and one or more pharmaceutical excipients may be granulated before being filled into a capsule or compressed into a tablet. The granulation process may be a dry or wet granulation process. The dry granulation process blends the drug with the one or more excipients, without the aid of a granulation fluid, to create agglomerates of the drug and one or more excipients. The dry granulation process may employ the application of force such as roller compaction or slugging to create the agglomerates. The wet granulation process blends the drug with the one or more excipients in the presence of a granulation fluid to create agglomerates of the drug and one or more excipients.

Some solid dosage forms contain drugs subject to abuse, such as opioids, which may be abused by crushing and snorting or smoking the crushed tablet material, opening a capsule and snorting or smoking the powder content of the capsule, extracting the drug from the tablet or capsule material with water or alcohol and injecting the extracted drug, or ingesting the tablet or capsule with alcohol to achieve a dose dumping. Accordingly, a need exists in the art for a dosage form capable of preventing or deterring diversion by any of the methods noted above.

Moreover, solid oral dosage forms containing drugs such as opioids are commonly formulated for extended release in order to provide pain relief over an extended time period. There are different kinds of drug delivery systems with various drug release control mechanisms that have been developed, including matrix tablets, film-coated tablets, press-coated tablets, multi-particulates and coated beads, layered tablets, osmotic tablets, etc. Some of the commercially available dosage forms employing these prior art technologies include OXYCONTIN, an extended release oxycodone tablet; EMBEDA and AVINZA, extended release morphine sulfate capsules; EXALGO, an extended release hydromorphone tablet; OPANA ER, an extended release oxymorphone tablet; NUCYNTA ER, an extended release tapentadol tablet; ZOHYDRO ER, an extended release hydrocodone capsule; and HYSINGLA, an extended release hydrocodone tablet. Patents relating to these commercially available tablets include, but are not limited to, U.S. Pat. Nos. 6,066,339; 6,733,783; 7,682,633; 7,815,934; 8,192,722; 8,309,060; 8,647,667; 8,808,737; 8,808,741; 8,877,247; 9,084,816; 9,132,096; and 9,486,451 which are incorporated in their entirety herein by reference.

Some of the aforementioned products employ controlled release pellets or beads which may be incorporated into a capsule or compressed into a tablet. The controlled release pellets or beads, typically employ a drug containing core that is surrounded by a rate controlling membrane. The rate controlling membrane may be semi-permeable and allow fluids, such as water from a patient's gastro-intestinal tract, to permeate the rate controlling membrane and dissolve the drug in the core. The dissolved drug migrates through the rate controlling membrane and is released from the controlled release pellet or bead over an extended period of time. The rate at which the drug is released through the rate controlling membrane can be controlled by adjusting the thickness and/or composition of the membrane. For example, a thicker membrane provides for a slower release of the drug because the dissolved drug molecule has a longer path to traverse before being released. Similarly, if the membrane incorporates water soluble material, which will dissolve in aqueous environments such as a patient's gastro-intestinal tract, pores and pathways can form in the rate controlling membrane allowing for a quicker release profile.

Some of the aforementioned products employ a matrix technology, which combines the drug with a controlled release matrix forming material, typically a hydrophilic polymer that absorbs water when placed into an aqueous environment such as a patient's gastro-intestinal tract or a hydrophobic material. The mixture of the drug and controlled release matrix forming material can be formed into tablets. If a hydrophilic polymer is employed the drug is released from the matrix tablet through a process of hydration, diffusion, and erosion. For example, when a matrix tablet employing a hydrophilic polymer is placed in an aqueous environment, the water hydrates the hydrophilic polymer creating a gel layer through which the drug must diffuse to be released. The gel layer also erodes over time. The thickness, rate of hydration, and erosion rate of the gel layer contribute to the controlled or sustained release of the drug from the matrix tablet. By varying the type and amount of hydrophilic polymer employed in a matrix tablet, the drug release rate can be adjusted to obtain a desired release rate. For example, a matrix tablet employing a large amount of a high molecular weight hydrophilic polymer will provide for a slower drug release compared to a similar matrix tablet employing less hydrophilic polymer and/or a hydrophilic polymer with a lower molecular weight.

In traditional matrix tablets, the drug and controlled release matrix forming material are either directly mixed and compressed into a tablet or the drug and controlled release matrix forming material are granulated and compressed into tablets. The granulation process may be a dry or wet granulation process as previously described. The use of hydrophilic or gelling polymers in the wet granulation process is difficult because the granulation fluid could cause the hydrophilic polymer to hydrate and gel resulting in an unworkable mass.

### SUMMARY OF THE INVENTION

The present invention relates to solid dosage forms that may exhibit an immediate or modified release of the drug from the solid dosage form when administered to a patient or tested using known in vitro testing apparatus such as those described in the United States Pharmacopeia section <711> Dissolution and <724> Drug Release. The solid dosage forms of the present invention may also exhibit abuse deterrent properties.

The solid dosage forms of the present invention comprise a plurality of drug-loaded particles. The drug-loaded particles comprise a core and a drug-load. The core is a hydrophilic polymer and the drug-load comprises a drug and a binder applied to the core.

The drug-load is applied to the core via a drug-loading solution, suspension, or dispersion and the drug-load may partially or completely surround the core. In one embodiment, the drug-loading solution, suspension, or dispersion comprises the drug, a binder, and a solvent and is applied onto the core via a spraying process, e.g., using a fluidized bed apparatus, a pan coating apparatus, or high shear granulating apparatus equipped with a spray nozzle. The drug-loading solution, suspension, or dispersion may also comprise conventional pharmaceutical excipients to aid in the manufacture and processing such as a lubricant, glidant, antioxidant, stabilizer, plasticizer, etc. In certain embodiments the drug-loading solution, suspension, or dispersion is created so that after application onto the core, a film containing the drug and binder is present on the core.

The core is a hydrophilic or gelling material, preferably a gelling polymeric material with an average particle size of about 20 μm to 900 μm, preferably about 30 μm to 500 μm and most preferably about 75 μm to about 300 μm. The core material may be irregularly shaped or regularly shaped, e.g., spherical. In certain embodiments the core is irregularly shaped, meaning that the core material is not spherical or uniform and in certain applications, the core is a hydrophilic or gelling polymer that is used in the same shape and form provided by the manufacturer or supplier.

The solid dosage form of the present invention may be an immediate release tablet, capsule or particulate dosage form wherein the particulate dosage form comprises a plurality of the drug-loaded particles in a container such as a sachet, pouch or vial. The solid dosage form of the present invention may be a modified release tablet, capsule or particulate dosage form. The release rate of the drug from the solid dosage forms of the present invention can be modified by: (i) adjusting the type the hydrophilic or gelling material employed in the core; (ii) adjusting the size of the core; (iii) adjusting the type and amount of binder employed to apply the drug-load to the core; (iv) adjusting amount of extra-granular material, i.e., excipients other than the drug-loaded particles, combined with the drug-loaded particles; and/or (v) type of final dosage form prepared with the drug-loaded particles. For example, a lower molecular weight gelling material for the core will allow for a faster drug release than a higher molecular weight gelling material. Similar, use of a water insoluble or pH dependent binder material to apply the drug to the core will delay the release of the drug compared to the use of a water soluble binder. Still further, compressing the drug-loaded particles into a tablet will slow the release of the drug compared to sprinkling the drug-loaded particles onto food from a sachet, pouch or vial.

Embodiments of the present invention may provide for a controlled or sustained release of the drug over an extended period of time following administration to a patient. In certain embodiments, the drug is released over a period of time from about 6 to about 24 hours, preferably about 8 to about 24 hours, and most preferably about 12 to about 24 hours. The dosage forms of the present invention allow for once, twice, or thrice a day dosing.

Embodiments of the present invention may exhibit abuse-deterrent properties such as preventing or hindering dose dumping when administered with alcohol; preventing or hindering the crushing and/or breaking by hand or hitting with a solid object such as a hammer; preventing or hindering the extraction of the drug into a needle for injection even if the dosage form has been manipulated such as subjected to grinding or a combination thereof.

In certain embodiments, the drug-loaded particles of the invention may be placed into a capsule or compressed into a tablet. In certain embodiments, the drug-loaded particles of the present invention may be combined with extra-granular excipients, i.e., the extra-granular component, prior to loading into a capsule or being compressed into a tablet. Depending on the amount and type of extra-granular excipients, the release rate of the drug from the final dosage form can be adjusted to a desired profile. For example, the addition of a water soluble filler such as lactose or mannitol to the drug-loaded particles can increase the release rate of the drug from a tablet or capsule prepared with the drug-loaded particles.

In certain embodiments, the present invention is also directed to a solid dosage form that employs a plurality of drug-loaded particles that comprise a core of a first hydrophilic polymer and a drug-load, and an extra-granular component that includes a second hydrophilic polymer and optionally at least one pharmaceutical excipient such as, a filler, an antioxidant, a glidant, a lubricant or combination thereof. The combination of the drug-loaded particles, extra-granular component and optional pharmaceutical excipient may be compressed into a tablet, filled into a capsule, sachet, pouch or vial.

In certain embodiments, the present invention is also directed to a solid dosage form that employs a plurality of drug-loaded particles optionally at least one pharmaceutical excipient such as, a filler, an antioxidant, a glidant, a lubricant or combination thereof. In this embodiment, the drug-loaded particles and optional one pharmaceutical excipient may be compressed into a tablet or filled into a capsule, sachet, pouch or vial. In this embodiment, the drug-loaded particles may preferably comprise at least 50 wt % or more of the solid dosage form, at least 75 wt % or more of the solid dosage form, at least 80 wt % or more of the solid dosage form, at least 85 wt % or more of the solid dosage form, at least 90 wt % or more of the solid dosage form, at least 95 wt % or more of the solid dosage form, at least 97 wt % or more or the solid dosage form, at least 99 wt % or more of the solid dosage form (excluding the weight of the capsule or container, i.e. pouch or vial). In one embodiment the solid dosage form consists of the drug-loaded particles and optionally a lubricant and/or glidant loaded into a capsule.

The present invention is additionally directed to a process for preparing a solid dosage form including the steps of:
preparing a drug-loading solution, suspension, or dispersion comprising: (a) a drug; (b) a binder which in certain embodiments may be selected from a hydrophilic polymer, a water soluble polymer, a hydrophobic polymer, a water-insoluble polymer or a combination thereof; (c) a solvent; and optionally (d) at least one additional pharmaceutical excipient;
(ii) spraying the drug-loading solution, suspension, or dispersion onto particles of a first hydrophilic polymer to form drug-loaded particles;
(iii) drying the drug-loaded particles;
(iv) optionally blending the drug-loaded particles with an extra-granular composition which may include a second hydrophilic polymer and/or at least one additional pharmaceutical excipient to form a final blend; and
(v) forming the drug-loaded particles into a solid dosage form.

Embodiments of the present invention are further directed to a methods of treating disease or symptoms of disease such as pain, hypertension, arrhythmia, hyperlipidemia, hypoglycemia, seizures, infections, psychosis, anxiety, or stress by administering to a patient in need of such treatment a solid dosage form comprising a plurality of the drug-loaded particles.

### BRIEF DESCRIPTION OF THE DRAWINGS

FIG. 1 depicts a drug-loaded particle in accordance with the present invention. The image on the left depicts intact drug-loaded particles prepared in accordance with the procedure described in Example 1; the image on the right depicts a view of a drug-loaded particle with a drug-loaded layer and a loose polymer particle core.

FIG. 2 depicts a compressed tablet in accordance with the present invention. The image on the left depicts the surface of the tablet; the image on the right depicts a section view of bound drug-loaded granules in the tablet.

FIG. 3 shows a representative in vitro dissolution profile of a tablet prepared in accordance with the present invention as described in Example 1a.

FIG. 4 shows a representative in vitro dissolution profile of a tablet in accordance with the present invention in Example 2a.

FIG. 5 shows a representative in vitro dissolution profile of a capsule in accordance with the present invention in Example 3a.

FIG. 6 shows a representative in vitro dissolution profile of a tablet in accordance with the present invention in Example 4a.

FIG. 7 shows a representative in vitro dissolution profile of a tablet prepared in accordance with the present invention as described in Example 5.

FIG. 8 shows representative in vitro dissolution profiles of tablets in accordance with the present invention in Example 6.

FIG. 9 shows representative in vitro dissolution profiles of tablets in accordance with the present invention in Example 7.

FIG. 10 shows a representative in vitro dissolution profile of a tablet in accordance with the present invention in Example 8a.

FIG. 11 shows a representative in vitro dissolution profile of a tablet prepared in accordance with the present invention as described in Example 9a.

FIG. 12 shows representative in vitro dissolution profiles of tablets in accordance with the present invention in Example 10a.

FIG. 13 shows a representative in vitro dissolution profile of a tablet prepared in accordance with the present invention as described in Example 11a.

FIG. 14 shows representative in vitro dissolution profiles of tablets in accordance with the present invention in Example 12a.

FIG. 15 shows a representative in vitro dissolution profile of a capsule in accordance with the present invention in Example 13a.

FIG. 16 shows a representative in vitro dissolution profile of a capsule in accordance with the present invention in Example 14a.

FIG. 17 depicts a drug granules of Example 15b.

FIG. 18 depicts a drug-loaded particle of Example 15c.

FIG. 19 shows a representative in vitro dissolution profile of the tablets prepared Examples 15a, 15b, and 15c.

FIG. 20 shows the in vitro dissolution profile of the capsules of Examples 15a, 15b, and 15c.

FIG. 21 shows the particle size distribution of the tablets of Examples 15a, 15b, and 15c after grinding.

FIG. 22 shows the in vitro dissolution profile of Example 16.

FIG. 23 shows the in vitro dissolution profile of Example 17.

FIG. 24 shows the in vitro dissolution profile of the tablets of Example 18a.

FIG. 25 shows the in vitro dissolution profile of the tablets of Example 19a.

### DETAILED DESCRIPTION

Except where noted, all terms are intended to have their normal meaning in the art, and are used as they would have been used by a person of ordinary skill at the time of the disclosure. It should be understood that throughout this application the singular forms, such as “a,” “an,” and “the,” are often used for convenience, however, these singular forms are intended to encompass the plural unless otherwise specified, or unless the context clearly calls for the singular alone.

“About” means having a value that is sufficiently close to the reference value so as to have identical or substantially identical properties as the reference value. Thus, depending on context, “about” can mean, for example, ±10%, ±9%, ±8%, ±7%, ±6, ±5%, ±4%, ±3%, ±2%, ±1%, or ±less than 1%.

“Pharmaceutically acceptable” refers to a material or method that can be used in medicine or pharmacy, including for veterinary purposes, for example, in administration to a subject.

“Salt” and “pharmaceutically acceptable salt” includes both acid and base addition salts. “Acid addition salt” refers to those salts that retain the biological effectiveness and properties of the free bases, which are not biologically or otherwise undesirable, and which are formed with inorganic acids and organic acids. “Base addition salt” refers to those salts that retain the biological effectiveness and properties of the free acids, which are not biologically or otherwise undesirable, and which are prepared from addition of an inorganic base or an organic base to the free acid.

“Treating” includes ameliorating, mitigating, and reducing the instances of a disease or condition, or the symptoms of a disease or condition. Because the instances of many diseases or conditions can be reduced before the disease or condition manifests, treating can also include prophylaxis.

“Administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound. “Administering” can also include providing directions to carry out a method involving a particular compound or a dosage form comprising the compound.

“Immediate Release” refers to a dosage form or component thereof that releases, or delivers about 100% of the one or more pharmaceutical agents or drugs in one hour or less, preferably in 45 minutes or less and most preferably in 30 minutes or less following administration to a subject or when tested using known in vitro testing apparatus such as those described in the United States Pharmacopeia section <711> Dissolution and <724> Drug Release.

“Modified release” refers to a dosage form or component thereof that releases, or delivers one or more pharmaceutical agents or drugs in a manner other than immediate release. Modified release includes, pulsatile release, delayed release, controlled release, sustained release, extended release or a combination thereof.

“Delayed release” refers to a dosage form or component thereof that releases, or delivers one or more pharmaceutical agents or drugs after a predetermine period of time following administration to a subject or when tested using known in vitro testing apparatus such as those described in the United States Pharmacopeia section <711> Dissolution and <724> Drug Release. Examples of delayed release dosage forms include dosage forms that release or deliver the pharmaceutical agent or drug when placed into a specific pH environment, i.e. a pH dependent release such as an enteric coated tablet or capsule.

“Controlled release,” (also known as CR), “Sustained release,” (also known as SR), and “Extended release” (also known as ER), are used synonymously herein and refer to a pharmaceutical formulation or component thereof that releases, or delivers, one or more pharmaceutical agents or drugs over a prolonged period of time, such as over 4 hours, 6 hours, 8 hours, 10 hours, 12 hours, 14 hours, 16 hours, 18 hours, 20 hours, or 24 hours following administration to a subject or when tested using known in vitro testing apparatus such as those described in the United States Pharmacopeia section <711> Dissolution and <724> Drug Release.

Drugs that may be used in the present invention include but are not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid suppressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor. Examples of the drugs in each of the foregoing classes can be found in the literature such as Remington, The Science and Practice of Pharmacy, 22^{nd} ed. (2013) and Goodman & Gilman's, The Pharmacological Basis of Therapeutics, 9^{th} ed. (1996), which are incorporated herein by reference.

Examples of drugs that may be employed in the present invention include:

Analgesic agents such as: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Loreinadol; Lornoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propiram Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

Anesthetic agents such as: Aliflurane; Benoxinate Hydrochloride; Benzocaine; Biphenamine Hydrochloride; Bupivacaine Hydrochloride; Butamben; Butamben Picrate; Chloroprocaine Hydrochloride; Cocaine; Cocaine Hydrochloride; Cyclopropane; Desflurane; Dexivacaine; Diamocaine Cyclamate; Dibucaine; Dibucaine Hydrochloride; Dyclonine Hydrochloride; Enflurane; Ether; Ethyl Chloride; Etidocaine; Etoxadrol Hydrochloride; Euprocin Hydrochloride; Fluroxene; Halothane; Isobutamben; Isoflurane; Ketamine Hydrochloride; Levoxadrol Hydrochloride; Lidocaine; Lidocaine Hydrochloride; Mepivacaine Hydrochloride; Methohexital Sodium; Methoxyflurane; Midazolam Hydrochloride; Midazolam Maleate; Minaxolone; Nitrous Oxide; Norflurane; Octodrine; Oxethazaine; Phencyclidine Hydrochloride; Pramoxine Hydrochloride; Prilocaine Hydrochloride; Procaine Hydrochloride; Propanidid; Proparacaine Hydrochloride; Propofol; Propoxycaine Hydrochloride; Pyrrocaine; Risocaine; Rodocaine; Roflurane; Salicyl Alcohol; Sevoflurane; Teflurane; Tetracaine; Tetracaine Hydrochloride; Thiamylal; Thiamylal Sodium; Thiopental Sodium; Tiletamine Hydrochloride; Zolamine Hydrochloride.

Antagonist agents such as: Atipamezole; Atosiban; Bosentan; Cimetidine; Cimetidine Hydrochloride; Clentiazem Maleate; Detirelix Acetate; Devazepide; Donetidine; Etintidine Hydrochloride; Famotidine; Fenmetozole Hydrochloride; Flumazenil; Icatibant Acetate; Icotidine; Isradipine; Metiamide; Nadide; Nalmefene; Nalmexone Hydrochloride; Naloxone Hydrochloride; Naltrexone; Nilvadipine; Oxilorphan; Oxmetidine Hydrochloride; Oxmetidine Mesylate; Quadazocine Mesylate; Ranitidine; Ranitidine Bismuth Citrate; Ranitidine Hydrochloride; Sufotidine; Teludipine Hydrochloride; Tiapamil Hydrochloride; Tiotidine; Vapiprost Hydrochloride; Zaltidine Hydrochloride.

Anti-anginal agents such as: Amlodipine Besylate; Amlodipine Maleate; Betaxolol Hydrochloride; Bevantolol Hydrochloride; Butoprozine Hydrochloride; Carvedilol; Cinepazet Maleate; Metoprolol Succinate; Molsidomine; Monatepil Maleate; Primidolol; Ranolazine Hydrochloride; Tosifen; Verapamil Hydrochloride.

Anti-anxiety agents such as: Adatanserin Hydrochloride; Alpidem; Binospirone Mesylate; Bretazenil; Glemanserin; Ipsapirone Hydrochloride; Mirisetron Maleate; Ocinaplon; Ondansetron Hydrochloride; Panadiplon; Pancopride; Pazinaclone; Scrazaipine Hydrochloride; Tandospirone Citrate; Zalospirone Hydrochloride.

Anticholinergic agents such as: Alverinc Citrate; Anisotropine Methylbromide; Atropine; Atropine Oxide Hydrochloride; Atropine Sulfate; Belladonna; Benapryzine Hydrochloride; Benzetimide Hydrochloride; Benzilonium Bromide; Biperiden; Biperiden Hydrochloride; Biperiden Lactate; Clidinium Bromide; Cyclopentolate Hydrochloride; Dexetimide; Dicyclomine Hydrochloride; Dihexyverine Hydrochloride; Domazoline Fumarate; Elantrine; Elucaine; Ethybenztropine; Eucatropine Hydrochloride; Glycopyrrolate; Heteronium Bromide; Homatropine Hydrobromide; Homatropine Methylbromide; Hyoscyamine; Hyoscyamine Hydrobromide; Hyoscyamine Sulfate; Isopropamide Iodide; Mepenzolate Bromide; Methylatropine Nitrate; Metoquizine; Oxybutynin Chloride; Parapenzolate Bromide; Pentapiperium Methyl sulfate; Phencarbamide; Poldine Methyl sulfate; Proglumide; Propantheline Bromide; Propenzolate Hydrochloride; Scopolamine Hydrobromide; Tematropium Methyl sulfate; Tiquinamide Hydrochloride; Tofenacin Hydrochloride; Toquizine; Triampyzine Sulfate; Trihexyphenidyl Hydrochloride; Tropicamide.

Anticonvulsant agents such as: Albutoin; Ameltolide; Atolide; Buramate; Carbamazepine; Cinromide; Citenamide; Clonazepam; Cyheptamide; Dezinamide; Dimethadione; Divalproex Sodium; Eterobarb; Ethosuximide; Ethotoin; Flurazepam Hydrochloride; Fluzinamide; Fosphenytoin Sodium; Gabapentin; Ilepeimide; Lamotrigine; Magnesium Sulfate; Mephenytoin; Mephobarbital; Methetoin; Methsuximide; Milacemide Hydrochloride; Nabazenil; Nafimidone Hydrochloride; Nitrazepam; Phenacemide; Phenobarbital; Phenobarbital Sodium; Phensuximide; Phenytoin; Phenytoin Sodium; Primidone; Progabide; Ralitoline; Remacemide Hydrochloride; Ropizine; Sabeluzole; Stiripentol; Sulthiame; Thiopental Sodium; Tiletamine Hydrochloride; Topiramate; Trimethadione; Valproate Sodium; Valproic Acid; Vigabatrin; Zoniclezole Hydrochloride; Zonisamide.

Antidepressant agents such as: Adatanserin Hydrochloride; Adinazolam; Adinazolam Mesylate; Alaproclate; Aletamine Hydrochloride; Amedalin Hydrochloride; Amitriptyline Hydrochloride; Amoxapine; Aptazapine Maleate; Azaloxan Fumarate; Azepindole; Azipramine Hydrochloride; Bipenamol Hydrochloride; Bupropion Hydrochloride; Butacetin; Butriptyline Hydrochloride; Caroxazone; Cartazolate; Ciclazindol; Cidoxepin Hydrochloride; Cilobamine Mesylate; Clodazon Hydrochloride; Clomipramine Hydrochloride; Cotinine Fumarate; Cyclindole; Cypenamine Hydrochloride; Cyprolidol Hydrochloride; Cyproximide; Daledalin Tosylate; Dapoxetine Hydrochloride; Dazadrol Maleate; Dazepinil Hydrochloride; Desipramine Hydrochloride; Dexamisole; Deximafen; Dibenzepin Hydrochloride; Dioxadrol Hydrochloride; Dothiepin Hydrochloride; Doxepin Hydrochloride; Duloxetine Hydrochloride; Eclanamine Maleate; Encyprate; Etoperidone Hydrochloride; Fantridone Hydrochloride; Fenmetozole Hydrochloride; Fenmetramide; Fezolamine Fumarate; Fluotracen Hydrochloride; Fluoxetine; Fluoxetine Hydrochloride; Fluparoxan Hydrochloride; Gamfexine; Guanoxyfen Sulfate; Imafen Hydrochloride; Imiloxan Hydrochloride; Imipramine Hydrochloride; Indeloxazine Hydrochloride; Intriptyline Hydrochloride; Iprindole; Isocarboxazid; Ketipramine Fumarate; Lofepramine Hydrochloride; Lortalamine; Maprotiline; Maprotiline Hydrochloride; Melitracen Hydrochloride; Milacemide Hydrochloride; Minaprine Hydrochloride; Mirtazapine; Moclobemide; Modaline Sulfate; Napactadine Hydrochloride; Napamezole Hydrochloride; Nefazodone Hydrochloride; Nisoxetine; Nitrafudam Hydrochloride; Nomifensine Maleate; Nortriptyline Hydrochloride; Octriptyline Phosphate; Opipramol Hydrochloride; Oxaprotiline Hydrochloride; Oxypertine; Paroxetine; Phenelzine Sulfate; Pirandamine Hydrochloride; Pizotyline; Pridefine Hydrochloride; Prolintane Hydrochloride; Protriptyline Hydrochloride; Quipazine Maleate; Rolicyprine; Seproxetine Hydrochloride; Sertraline Hydrochloride; Sibutramine Hydrochloride; Sulpiride; Suritozole; Tametraline Hydrochloride; Tampramine Fumarate; Tandamine Hydrochloride; Thiazesim Hydrochloride; Thozalinone; Tomoxetine Hydrochloride; Trazodone Hydrochloride; Trebenzomine Hydrochloride; Trimipramine; Trimipramine Maleate; Venlafaxine Hydrochloride; Viloxazine Hydrochloride; Zimeldine Hydrochloride; Zometapine.

Antidiabetic agents such as: Acetohexamide; Buformin; Butoxamine Hydrochloride; Caniglibose; Chlorpropamide; Ciglitazone; Englitazone Sodium; Etoformin Hydrochloride; Gliamilide; Glibornuride; Glicetanile Sodium; Gliflumide; Glipizide; Glucagon; Glyburide; Glyhexamide; Glymidine Sodium; Glyoctamide; Glyparamide; Insulin; Insulin, Dalanated; Insulin Human; Insulin Human, Isophane; Insulin Human Zinc; Insulin Human Zinc, Extended; Insulin, Isophane; Insulin Lispro; Insulin, Neutral; Insulin Zinc; Insulin Zinc, Extended; Insulin Zinc, Prompt; Linogliride; Linogliride Fumarate; Metformin; Methyl Palmoxirate; Palmoxirate Sodium; Pioglitazone Hydrochloride; Pirogliride Tartrate; Proinsulin Human; Seglitide Acetate; Tolazamide; Tolbutamide; Tolpyrramide; Troglitazone; Zopolrestat.

Anti-epileptic agents such as: Felbamate; Loreclezole; Tolgabide, Lamotrigine.

Antihistaminic agents such as: Acrivastine; Antazoline Phosphate; Astemizole; Azatadine Maleate; Barmastine; Bromodiphenhydramine Hydrochloride; Brompheniramine Maleate; Carbinoxamine Maleate; Cetirizine Hydrochloride; Chlorpheniramine Maleate; Cinnarizine; Clemastine; Clemastine Fumarate; Closiramine Aceturate; Cycliramine Maleate; Cyclizine; Cyproheptadine Hydrochloride; Dexbrompheniramine Maleate; Dexchlorpheniramine Maleate; Dimethindene Maleate; Diphenhydramine Citrate; Diphenhydramine Hydrochloride; Dorastine Hydrochloride; Doxylamine Succinate; Ebastine; Levocabastine Hydrochloride; Loratadine; Mianserin Hydrochloride; Noberastine; Orphenadrine Citrate; Pyrabrom; Pyrilamine Maleate; Pyroxamine Maleate; Rocastine Hydrochloride; Rotoxamine; Tazifylline Hydrochloride; Temelastine; Terfenadine; Tripelennamine Citrate; Tripelennamine Hydrochloride; Triprolidine Hydrochloride; Zolamine Hydrochloride.

Antihyperlipidemic agents such as: Cholestyramine Resin; Clofibrate; Colestipol Hydrochloride; Crilvastatin; Dalvastatin; Dextrothyroxine Sodium; Fluvastatin Sodium; Gemfibrozil; Lecimibide; Lovastatin; Niacin; Pravastatin Sodium; Probucol; Simvastatin; Tiqueside; Xenbucin.

Antihyperlipoproteinemic agents such as: Acifran; Beloxamide; Bezafibrate; Boxidine; Butoxamine Hydrochloride; Cetaben Sodium; Ciprofibrate; Gemcadiol; Halofenate; Lifibrate; Meglutol; Nafenopin; Pimetine Hydrochloride; Theofibrate; Tibric Acid; Treloxinate. Antihypertensive: Alfuzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydroflumethiazide; Indacrinone; Indapamide; Indolapril Hydrochloride; Indoramin; Indoramin Hydrochloride; Indorenate Hydrochloride; Lacidipine; Leniquinsin; Leveromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nifidipine; Nitrendipine; Ofornine; Pargyline Hydrochloride; Pazoxide; Pelanserin Hydrochloride; Perindopril Erbumine; Phenoxybenzamine Hydrochloride; Pinacidil; Pivopril; Polythiazide; Prazosin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine.

Anti-inflammatory agents such as: Alclofenae; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prednisolone Sodium Phosphate; Prodolic Acid; Proquazone; Proxazole; Proxazole Citrate; Rimexolone; Romazarit; Salcolex; Salnacediin; Salsalate; Sanguinarium Chloride; Seelzone; Sermetacin; Sudoxicam; Sulinldac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Antimigraine agents such as: Dolasetron Mesylate; Naratriptan Hydrochloride; Sergolexole Maleate; Sumatriptan Succinate; Zatosetron Maleate.

Antiparkinsonian agents such as: Benztropine Mesylate; Biperiden; Biperiden Hydrochloride; Biperiden Lactate; Carbidopa-Levodopa; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinielorane Hydrochloride; Ropinirole Hydrochloride; Selegiline Hydrochloride; Tolcapone; Trihexyphenidyl Hydrochloride.

Antipsychotic agents such as: Acetophenazine Maleate; Alentemol Hydrobromide; Alpertine; Azaperone; Batelapine Maleate; Benperidol; Benzindopyrine Hydrochloride; Brofoxine; Bromperidol; Bromperidol Decanoate; Butaclamol Hydrochloride; Butaperazine; Butaperazine Maleate; Carphenazine Maleate; Carvotroline Hydrochloride; Chlorpromazine; Chlorpromazine Hydrochloride; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; Droperidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Haloperidol Decanoate; Iloperidone; Imidoline Hydrochloride; Lenperone; Mazapertine Succiniate; Mesoridazine; Mesoridazine Besylate; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacetazine; Pipotiazine Palmitate; Piquindone Hydrochloride; Prochlorperazine Edisylate; Prochlorperazine Maleate; Promazine Hydrochloride; Remoxipride; Remoxipride Hydrochloride; Rimcazole Hydrochloride; Seperidol Hydrochloride; Sertindole; Setoperone; Spiperone; Thioridazine; Thioridazine Hydrochloride; Thiothixene; Thiothixene Hydrochloride; Tioperidone Hydrochloride; Tiospirone Hydrochloride; Trifluoperazine Hydrochloride; Trifluperidol; Triflupromazine; Triflupromazine Hydrochloride; Ziprasidone Hydrochloride.

Antithrombotic agents such as: Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; Trifenagrel.

Antitussive agents such as: Benzonatate; Butamirate Citrate; Chlophedianol Hydrochloride; Codeine; Codoxime; Dextromethorphan; Dextromethorphan Hydrobromide; Ethyl Dibunate; Guaiapate; Hydrocodone Bitartrate; Levopropoxyphene Napsylate; Noscapine; Pemerid Nitrate; Pipazethate; Suxemerid Sulfate.

Appetite suppressant agents such as: Dexfenfluramine Hydrochloride; Phendimetrazine Tartrate; Phentermine Hydrochloride.

Benign prostatic hyperplasia therapy agents such as: Tamsulosin Hydrochloride.

Blood glucose regulating agents such as: Human insulin; Glucagon; Tolazamide; Tolbutamide; Chloropropamide; Acetohexamide and Glipizide.

Cholinergic agents such as: Aceclidine; Bethanechol Chloride; Carbachol; Demecarium Bromide; Dexpanthenol; Echothiophate Iodide; Isoflurophate; Methacholine Chloride; Neostigmine Bromide; Neostigmine Methyl sulfate; Physostigmine; Physostigmine Salicylate; Physostigmine Sulfate; Pilocarpine; Pilocarpine Hydrochloride; Pilocarpine Nitrate; Pyridostigmine Bromide.

Diuretic agents such as: Ambuphylline; Ambuside; Amiloride Hydrochloride; Azolimine; Azosemide; Brocrinat; Bumetanide; Chlorothiazide; Chlorthalidone; Clazolimine; Clorexolone; Ethacrynate Sodium; Ethacrynic Acid; Etozolin; Fenquizone; Furosemide; Hydrochlorothiazide; Isosorbide; Mannitol; Mefruside; Ozolinone; Piretanide; Spiroxasone; Torsemide; Triamterene; Triflocin; Urea.

Emetic agents such as: Apomorphine Hydrochloride.

HMGCoA reductase inhibiting agents such as: Lovastatin; Simvastatin; Pravastatin; Fluvasatin; Atorvastatin; Rosuvastatin.

Nasal decongestant agents such as: Nemazoline Hydrochloride; Pseudoephedrine.

Relaxant agents such as: Adipheinine Hydrochloride; Aleuronium Chloride; Aminophylline; Azumolene Sodium; Baclofen; Benzoctamine Hydrochloride; Carisoprodol; Chlorphenesin Carbamate; Chlorzoxazone; Cinflumide; Cinnamedrine; Clodanolene; Cyclobenzaprine Hydrochloride; Dantrolene; Dantrolene Sodium; Fenalamide; Fenyripol Hydrochloride; Fetoxylate Hydrochloride; Flavoxate Hydrochloride; Fletazepam; Flumetramide; Flurazepam Hydrochloride; Hexafluorenium Bromide; Isomylamine Hydrochloride; Lorbamate; Mebeverine Hydrochloride; Mesuprine Hydrochloride; Metaxalone; Methocarbamol; Methixene Hydrochloride; Nafomine Malate; Nelezaprine Maleate; Papaverine Hydrochloride; Pipoxolan Hydrochloride; Quinctolate; Ritodrine; Ritodrine Hydrochloride; Rolodine; Theophylline Sodium Glycinate; Thiphenamil Hydrochloride; Xilobam.

Sedative-hypnotic agents such as: Allobarbital; Alonimid; Alprazolam; Amobarbital Sodium; Bentazepam; Brotizolam; Butabarbital; Butabarbital Sodium; Butalbital; Capuride; Carbocloral; Chloral Betaine; Chloral Hydrate; Chlordiazepoxide Hydrochloride; Cloperidone Hydrochloride; Clorethate; Cyprazepam; Dexclamol Hydrochloride; Diazepam; Dichloralphenazone; Estazolam; Ethehlorvynol; Etomidate; Fenobam; Flunitrazepam; Fosazepam; Glutethimide; Halazepam; Lormetazepam; Mecloqualone; Meprobamate; Methaqualone; Midaflur; Paraldehyde; Pentobarbital; Pentobarbital Sodium; Perlapine; Prazepam; Quazepam; Reclazepam; Roletamicide; Secobarbital; Secobarbital Sodium; Suproclone; Thalidomide; Tracazolate; Trepipam Maleate; Triazolam; Tricetamide; Triclofos Sodium; Trimetozine; Uldazepam; Zaleplon; Zolazepam Hydrochloride; Zolpidem Tartrate.

Stimulant agents such as: Amfonelic Acid; Amphetamine Sulfate; Ampyzine Sulfate; Arbutamine Hydrochloride; Azabon; Caffeine; Ceruletide; Ceruletide Diethylamine; Cisapride; Dazopride Fumarate; Dextroamphetamine; Dextroamphetamine Sulfate; Difluanine Hydrochloride; Dimefline Hydrochloride; Doxapram Hydrochloride; Etryptamine Acetate; Ethamivan; Fenethylline Hydrochloride; Flubanilate Hydrochloride; Flurothyl; Histamine Phosphate; Indriline Hydrochloride; Mefexamide; Methamphetamine Hydrochloride; Methylphenidate Hydrochloride; Pemoline; Pyrovalerone Hydrochloride; Xamoterol; Xamoterol Fumarate.

Tranquilizing agents such as: Bromazepam; Buspirone Hydrochloride; Chlordiazepoxide; Clazolam; Clobazam; Clorazepate Dipotassium; Clorazepate Monopotassium; Demoxepam; Dexmedetomidine; Enciprazine Hydrochloride; Gepirone Hydrochloride; Hydroxyphenamate; Hydroxyzine Hydrochloride; Hydroxyzine Pamoate; Ketazolam; Lorazepam; Lorzafone; Loxapine; Loxapine Succinate; Medazepam Hydrochloride; Nabilone; Nisobamate; Oxazepam; Pentabamate; Pirenperone; Ripazepam; Rolipram; Sulazepam; Taciamine Hydrochloride; Temazepam; Triflubazam; Tybamate; Valnoctamide.

Vasodilator agents such as: Alprostadil; Azaclorzine Hydrochloride; Bamethan Sulfate; Bepridil Hydrochloride; Buterizine; Cetiedil Citrate; Chromonar Hydrochloride; Clonitrate; Diltiazem Hydrochloride; Dipyridamole; Droprenilamine; Erythrityl Tetranitrate; Felodipine; Flunarizine Hydrochloride; Fostedil; Hexobendine; Inositol Niacinate; Iproxamine Hydrochloride; Isosorbide Dinitrate; Isosorbide Mononitrate; Isoxsuprine Hydrochloride; Lidoflazine; Mefenidil; Mefenidil Fumarate; Mibefradil Dihydrochloridc; Mioflazine Hydrochloride; Mixidine; Nafronyl Oxalate; Nicardipine Hydrochloride; Nicergoline; Nicorandil; Nicotinyl Alcohol; Nifedipine; Nimodipine; Nisoldipine; Oxfenicine; Oxprenolol Hydrochloride; Pentaerythritol Tetranitrate; Pentoxifylline; Pentrinitrol; Perhexiline Maleate; Pindolol; Pirsidomine, Prenylamine; Propatyl Nitrate; Suloctidil; Terodiline Hydrochloride; Tipropidil Hydrochloride; Tolazoline Hydrochloride; Xanthinol Niacinate.

In preferred embodiments, the drug employed in the solid dosage forms of the present invention is a water soluble drug and should exhibit a water solubility of at least about 1 mg per 100 ml of water, preferably at least about 1 mg per 10 ml of water and most preferably at least about 1 mg per 1 ml of water at 20° C.

Certain embodiments, the drug employed in the solid dosage forms or the present invention are water soluble drugs selected from the group of analgesic agents, anesthetic agents, anti-anginal agents, anti-anxiety agents, anti-convulsant agents, anti-depressants, anti-inflammatory agents, anti-psychotic agents, anti-tussive agents, appetite suppressant agents, relaxant agents, sedative-hypnotic agents, stimulants, tranquilizing agent and vasodilator agents as identified above.

Specific embodiments of the present invention employ one or more drugs that are BCS class I drugs. BCS refers to Biopharmaceutical Classification System (“BCS”) which classifies drugs into one of four categories according to the drug's solubility and permeability properties. The four class are: BCS class I drugs have a high permeability and high solubility; BCS class II drugs have a high permeability and low solubility; BCS class III drugs have a low permeability and high solubility; and BCS class IV drugs have a low permeability and a low solubility. The BCS system is further described in the United States Food and Drug Administration's May 2015 Draft Guidance for Industry, entitled “Waiver of In Vivo Bioavailability and Bioequivalence Studies for Immediate-Release Solid Oral Dosage Forms Based on a Biopharmaceutics Classification System” which is incorporated herein by reference. Examples of BCS class I drugs that may be used in the present invention include pegabalin, gabapentin, venlafaxine HCl, desvenlafaxine succinate, desvenlafaxine fumarate, methylphenidate HCl, dexmethylphenidate HCl, tramadol HCl, hydrocodone bitartrate, morphine sulfate, oxycodone HCl, amphetamine salts, metoprolol succinate and tapentadol HCl.

Specific embodiments of the present invention employ one or more drugs that are subject to abuse, such as opioids, opiates, stimulants, tranquilizers, and narcotics. In a preferred embodiment, the drug for use in the present dosage forms is one or more of 1-phenyl cyclohexylamine, 1-piperidinocyclohexanecarbonitrile, alfentanil, alphacetylmethadol, alphaprodine, alprazolam, amobarbital, amphetamine, anileridine, apomorphine, aprobarbital, barbital, barbituric acid derivative, bemidone, benzoylecgonine, benzphetamine, betacetylmethadol, betaprodine, bezitramide, bromazepam, buprenorphine, butabarbital, butalbital, butorphanol, camazepam, cathine, chloral, chlordiazepoxide, clobazam, clonazepam, clorazepate, clotiazepam, cloxazolam, cocaine, codeine, chlorphentermine, delorazepam, dexfenfluramine, dextromethorphan, dextromoramide, dextropropoxyphen, dezocine, diazepam, diethylpropion, difenoxin, dihydrocodeine, dihydromorphine, dioxaphentyl butyrate, dipanone, diphenoxylate, diprenorphine, ecgonine, enadoline, eptazocine, estazolam, ethoheptazine, ethyl loflazepate, ethylmorphine, etorphine, femproponex, fencamfamin, fenfluramine, fentanyl, fludiazepam, flunitrazepam, flurazepam, glutethimide, halazepam, haloxazolam, hexalgon, hydrocodone, hydromorphone, isomethadone, ketamine, ketazolam, ketobemidone, levanone, levoalphacetylmethadol, levomethadone, levomethadyl acetate, levomethorphan, levorphanol, lofentanil, loperamide, loprazolam, lorazepam, lormetazepam, lysergic acid, lysergic acid amide, mazindol, medazepam, mefenorex, meperidine, meptazinol, metazocine, methadone, methamphetamine, methohexital, methotrimeprazine, methyldihydromorphinone, methylphenidate, methylphenobarbital, metopon, morphine, nabilone, nalbuphine, nalbupine, nalorphine, narceine, nefopam, nicomorphine, nimetazepam, nitrazepam, nordiazepam, normethadone, normorphine, oxazepam, oxazolam, oxycodone, oxymorphone, pentazocine, pentobarbital, phenadoxone, phenazocine, phencyclidine, phendimetrazine, phenmetrazine, pheneridine, piminodine, prodilidine, properidine, propoxyphene, racemethorphan, racemorphan, racemoramide, remifentanil, secobarbital, sufentanil, talbutal, tapentadol, thebaine, thiamylal, thiopental, tramadol, trimeperidine, and vinbarbital, salts, derivatives, analogs, homologues, polymorphs thereof, and mixtures of any of the foregoing.

The solid dosage forms of the present invention may also employ one or more of allobarbitone, alprazolam, amylobarbitone, aprobarbital, barbital, barbitone, benzphetamnine, brallobarbital, bromazepam, brotizolam, buspirone, butalbital, butobarbitone, butorphanol, camazepam, captodiame, carbromal, carfentanil, carpipramine, cathine, chloral, chloral betaine, chloral hydrate, chloralose, chlordiazepoxide, chlorhexadol, chlormethiazole edisylate, chlormezanone, cinolazepam, clobazam, potassium clorazepate, clotiazepam, cloxazolam, cyclobarbitone, delorazepam, dexfenfluramine, diazepam, diethylpropion, difebarbamate, difenoxin, enciprazine, estazolam, ethyl loflazepate, etizolam, febarbamate, fencamfamin, fenfluramine, fenproporex, fluanisone, fludiazepam, flunitraam, flunitrazepam, flurazepam, flutoprazepam, gepirone, glutethimide, halazepam, haloxazolam, hexobarbitone, ibomal, ipsapirone, ketazolam, loprazolam mesylate, lorazepam, lormetazepam, mazindol, mebutamate, medazepam, mefenorex, mephobarbital, meprobamate, metaclazepam, methaqualone, methohexital, methylpentynol, methylphenobarbital, midazolam, milazolam, morphine, nimetazepam, nitrazepam, nordiazepam, oxazepam, oxazolam, paraldehyde, pemoline, pentabarbitone, pentazocine, pentobarbital, phencyclidine, phenobarbital, phendimetrazine, phenmetrazine, phenprobamate, phentermine, phenyacetone, pinazepam, pipradol, prazepam, proxibarbal, quazepam, quinalbaritone, secobarbital, secbutobarbitone, sibutramine, temazepam, tetrazepam, triazolam, triclofos, zalepan, zaleplon, zolazepam, zolpidem, and zopiclone, salts, derivatives, analogs, homologues, polymorphs thereof, and mixtures of any of the foregoing.

In a preferred embodiment, the drug used in the present invention is an opioid selected from morphine, hydrocodone, oxycodone, oxymorphone, and tapentadol, or combinations thereof or a pharmaceutically acceptable salt thereof, such as the hydrochloride salt.

In embodiments where the opioid includes morphine, the morphine or pharmaceutically acceptable salt thereof may be employed at about 2 mg to about 250 mg or about 5 mg to about 200 mg. In embodiments where the opioid includes oxycodone, the oxycodone or pharmaceutically acceptable salt thereof may be employed at about 2 mg to about 160 mg, about 5 mg to about 75 mg, about 5 mg to about 40 mg, or about 10 mg to about 30 mg. In embodiments where the opioid includes hydrocodone, the hydrocodone or pharmaceutically acceptable salt thereof may be employed at about 0.5 mg to about 1250 mg, about 5 mg to about 60 mg, about 15 mg to about 40 mg, or about 20 mg to about 30 mg. In embodiments where the opioid includes oxymorphone, the oxymorphone or pharmaceutically acceptable salt thereof may be employed at about 1 to about 100 mg, about 2.5 to about 75 mg, about 5 to about 50 mg, about 10 to about 40 mg, or about 20 to about 30 mg. In a preferred embodiment, the dosage form employs about 10 to about 40 mg or about 20 to about 30 mg of oxymorphone HCl. In embodiments where the opioid includes tapentadol, the tapentadol or pharmaceutically acceptable salt thereof may be employed at about 25 mg to about 1000 mg, about 50 mg to about 500 mg, about 100 mg to about 500 mg, or about 200 mg to about 300 mg. In a preferred embodiment, the dosage form employs about 50 to about 500 mg, about 100 mg to about 500 mg, or about 200 mg to about 300 mg of tapentadol HCl.

The solid dosage forms of the present invention may further comprise a second drug, preferably a second drug that is not subject to abuse. For example, if the solid dosage form comprises an opioid, i.e., a drug that is subject to abuse, the dosage form may comprise a second drug that is not subject to abuse such as an analgesic, e.g., aspirin, acetaminophen, or an NSAID such as naproxen, ibuprofen, or diclofenac.

In an embodiment, the amount of drug in the solid dosage form ranges from about 5% to about 90% by weight of the total weight of the solid dosage form, about 10% to about 80% by weight of the total weight of the solid dosage form, about 15% to about 75% by weight of the total weight of the solid dosage form, or about 20% to about 65% by weight of the total weight of the solid dosage form. In certain embodiments of the present invention, the amount of drug in the drug-loaded particles ranges from about 10% to about 80% by weight of the total weight of the drug-loaded particles, preferably about 15% to about 70% by weight of the total weight of the drug-loaded particles and most preferably about 20% to about 60% by weight of the total weight of the drug-loaded particles. In still further embodiments, the ratio of drug to core material in the drug-loaded particle ranges from about 0.1:1 to about 1:1, preferably about 0.25:1 to about 1:1, and most preferably about 0.5:1 to 1:1.

The drug-loaded particles employed in the solid dosage forms of the present invention are made by preparing a drug-loading solution, suspension or dispersion and applying the drug-loading solution, suspension or dispersion onto the core material. The drug-loading solution, suspension or dispersion comprises: (i) a drug; (ii) a binder which in certain embodiments may be (a) a hydrophilic polymer; (b) a water soluble polymer; (c) a hydrophobic polymer; (d) a water-insoluble polymer; or (e) a combination thereof; (iii) a solvent; and (iv) optionally at least one pharmaceutical excipient such as a lubricant, glidant, plasticizer, stabilizer, antioxidant, or combination thereof.

The drug-loading solution, suspension, or dispersion is applied onto a core material, which is preferably a hydrophilic or gelling polymer. Suitable hydrophilic or gelling polymers for use as the core material in the present invention include, without limitation, polyalkylene oxides, particularly poly(ethylene oxide), polyethylene glycol and poly(ethylene oxide)-poly(propylene oxide) copolymers; cellulosic polymers, such as methylcellulose, hydroxymethyl cellulose, hydroxyethyl cellulose, hydroxypropyl cellulose, hydroxypropyl methylcellulose, and carboxymethyl cellulose, microcrystalline cellulose, and polysaccharides and their derivatives; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); polyalkylene oxides; poly(olefinic alcohol)s such as poly(vinyl alcohol); poly(N-vinyl lactams) such as poly(vinyl pyrrolidone), poly(N-vinyl caprolactam), and copolymers thereof; polysaccharide gums such as xanthan gum, and guar gum, carrageenan, starches and alginates. Some of the preferred hydrophilic or gelling polymers that may be used as the core material for the present invention include but are not limited to polyethylene oxide, hydroxypropyl cellulose, hydroxypropyl methylcellulose, carbomers (i.e. acrylic acid polymers or Carbopol), carboxymethylcellulose calcium, carboxymethylcellulose sodium, croscarmellose sodium, carrageenan, sodium starch glycolate, xanthan gum, guar gum, sodium alginate, calcium alginate. These hydrophilic or gelling polymers may be used individually or in combination.

In one embodiment, the hydrophilic of gelling polymer(s) may be a cellulosic polymer, such as an alkyl substituted cellulose derivative. In one embodiment, the polymer is an alkyl substituted cellulose having a viscosity within the range of about 100 to about 110,000 centipoise as a 2% aqueous solution at 20° C. In another embodiment, the polymer is an alkyl substituted cellulose having a viscosity within the range of about 1,000 to about 4,000 centipoise as a 1% aqueous solution at 20° C. Preferred alkyl celluloses are hydroxypropyl cellulose and hydroxypropyl methylcellulose.

In one embodiment, the hydrophilic or gelling polymer(s) may be a polyalkylene oxide. In another aspect, the polyalkylene oxide may be polyethylene oxide. In an embodiment, the polyethylene oxide may have an approximate molecular weight between 100,000 Daltons (Da) to about 10,000,000 Da or about 500,000 Da to about 7,000,000 Da. In a further embodiment, the polyethylene oxide may have a molecular weight of approximately 600,000 Da, 700,000 Da, 800,000 Da, 900,000 Da, 1,000,000 Da, 2,000,000 Da, 3,000,000 Da, 4,000,000 Da, 5,000,000 Da, 6,000,000 Da, 7,000,000 Da, 8,000,000 Da 9,000,000 Da, or 10,000,000 Da.

Polyethylene oxide is sold under the trade name POLYOX by Dow Chemical. In one embodiment, the polyethylene oxide may be any grade of POLYOX or combination thereof including, without limitation, WSR N-10 (M.W. 100,000 Da), WSR N-80 (M.W. 200,000 Da), WSR N-750 (M.W. 300,000 Da), WSR N-3000 (M.W. 400,000 Da), WSR 205 (M.W. 600,000 Da), WSR 1105 (M.W. 900,000 Da), WSR N-12K (M.W. 1,000,000 Da), WSR N-60K (M.W. 2,000,000 Da), WSR-301 (M.W. 4,000,000 Da), WSR Coagulant (M.W. 5,000,000 Da), WSR-303 (M.W. 7,000,000 Da), WSR-308 (M.W. 8,000,000 Da), UCARFLOC Polymer 300 (M.W. 4,000,000 Da), UCARFLOC Polymer 302 (M.W. 5,000,000 Da), UCARFLOC Polymer 304 (M.W. 7,000,000 Da), and UCARFLOC Polymer 309 (M.W. 8,000,000 Da).

In an embodiment of the present invention, the hydrophilic or gelling polymer(s) that may be used as the core material include a low molecular weight polyethylene oxide having an average molecular weight less than or equal to 1,000,000 Da, preferably 900,000 Da, and a high molecular weight polyethylene oxide having an average molecular weight of greater than or equal to 2,000,000 Da, preferably greater than or equal to 4,000,000 Da, most preferably greater than or equal to 5,000,000 Da. In a particularly preferred embodiment, the high molecular weight polyethylene oxide has an average molecular weight of 7,000,000 Da.

In embodiments employing a hydrophilic or gelling polymer core comprising a high and low molecular weight polyethylene oxide, the ratio of high molecular weight polyethylene oxide (i.e., greater than 1,000,000 Da) to low molecular weight polyethylene oxide (i.e., less than 1,000,000 Da) may range from about 1:1 to about 4:1 by weight, about 1:1 to about 3:1 by weight, about 1:1 to about 2.5:1 by weight, or about 2:1 by weight.

The hydrophilic or gelling polymer employed as the core material should have an average particle size less than about 1,500 μm, less than about 1,000 μm, less than about 850 μm, less than about 750 μm, or less than about 500 μm. In an embodiment of the present invention, the hydrophilic or gelling polymer employed as the core material should have an average particle size of about 5 μm to about 1000 μm, about 15 μm to about 900 μm, about 25 μm to about 800 μm, about 50 μm to about 750 μm or about 75 μm to about 500 μm.

Examples of binders that may be used in the drug-loading solution, suspension or dispersion include, without limitation, starches (including corn starch and pregelatinized starch), gelatin, sugars (including sucrose, glucose, dextrose and lactose), polyethylene glycol, polyols, polyvinyl alcohols, C₁₂-C₁₈ fatty acid alcohols, waxes, gums (e.g., guar gum, arabic gum, acacia gum, xanthan gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, polymethacrylates, and polyvinyloxoazolidone, or combinations thereof. In preferred embodiments the binder may is (a) a hydrophilic polymer; (b) a water soluble polymer; (c) a hydrophobic polymer; (d) a water-insoluble polymer; or (e) a combination thereof.

Examples of preferred hydrophilic and/or water soluble polymers that may be used in the drug-loading solution include but are not limited to hydroxypropyl methylcellulose, hydroxypropyl cellulose, hydroxyethyl cellulose, povidone, copovidone, polyvinyl alcohol, polyglycols, pH dependent polymers and combinations thereof. Examples of pH dependent polymers include dimethyl amine methacrylates (commercially available under the tradename EUDRAGIT E), methacrylic acid copolymer (commercially available under the tradename EUDRAGIT L and EUDRAGIT S), hydroxypropyl methylcelluose acetate succinate, cellulose acetate phthalate, polyvinyl acetate phthalate and shellac. Embodiments employing a water soluble polymer as the drug-loading binder may preferably employ a low molecule weight water soluble polymer and most preferably water soluble polymer that exhibit a viscosity of less than 75 cps when a 2% aqueous solution is prepared preferably less than 50 cps and most preferably less than 30 cps. Some commercially available water soluble low molecular weight/low viscosity polymers include METHOCEL E3, METHOCEL E5, METHOCEL E6, MEHTOCEL E15, KLUCEL EF, KLUCEL LF, KLUCEL JF, HPC-SSL, HPC-SL, KOLLIDON K-30 and KOLLIDON-VA as well as commercially available coating materials containing the water soluble low molecular weight/low viscosity polymers such as the OPADRY brand products from Colorcon.

Examples of the hydrophobic and/or water-insoluble polymer that is employed as a binder in the drug-loading solution, suspension, or dispersion to apply the drug to the core material include, without limitation celluloses such as ethylcellulose, cellulose acetate, cellulose acetatebutyrate and polymethacrylates such as copolymers of ethyl acrylate and methyl methacrylate, and combinations thereof. In a preferred embodiment, the hydrophobic or water-insoluble film forming polymer is a polymethacrylate, preferably a copolymer of ethyl acrylate and methyl methacrylate, preferably in a 2:1 ratio. Examples of hydrophobic or water-insoluble film forming polymer are commonly sold under the trade name SURERELEASE, AQUACOAT, EUDRAGIT RS, EUDRAGIT RL. A preferred hydrophobic or water-insoluble film forming polymer is EUDRAGIT NE 30 D or EUDRAGIT NE 40 D.

In one embodiment, the amount of binder in the drug-loaded particles may range from about 0.5% to about 20% by weight of the drug-loaded particles, preferably about 1% to about 15% by weight of the drug-loaded particles, and most preferably about 2.0% to about 10% by weight of the drug-loaded-particles. In various embodiments, the amount of binder in the drug-loaded particles may be about 5.0%, 5.25%, 5.5%, 5.75%, 6.0%, 6.25%, 6.5%, 6.75%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, or 9.0% by weight of the drug-loaded particle.

The solvent for the drug-loading solution, suspension, or dispersion may be any conventional solvent used in the pharmaceutical arts. Such solvents include but are not limited to water, methanol, ethanol, isopropanol, acetone or a combination thereof.

The drug-load is applied to the material core via a drug-loading solution, suspension, or dispersion and the drug-load may partially or completely surround the core. In one embodiment, the drug-loading solution, suspension, or dispersion is applied onto the core via a spraying process, e.g., using a fluidized bed apparatus, a pan coating apparatus, or high shear granulating apparatus equipped with a spray nozzle. In an embodiment of the present invention the drug-load is applied to the core material in a fluidized bed process wherein the core material is fluidized and optionally heated in a fluid bed granulator prior to application of the drug-load coating. Once the core material is fluidized, the drug-loading solution, suspension, or dispersion is applied to the core material by spray coating the drug-loading solution, suspension, or dispersion, preferably using Wurster (bottom spray) technology. Once the drug-load is applied to the core materiel, the drug-loaded particles are dried, preferably in the fluid bed apparatus.

In certain embodiments, the drug-load may completely surround the core material, i.e., coat the core material. In another embodiment of the present invention, the drug-load only covers a portion of the core material, i.e., less than 95% of the surface area of the core material, less than 90% of the surface area of the core material, less than 85% of the surface area of the core material, less than 80% of the surface area of the core material, less than 75% of the surface area of the core material, less than 70% of the surface area of the core material, less than 65% of the surface area of the core material, less than 60% of the surface area of the core material, less than 55% of the surface area of the core material, or less than 50% of the surface area of the core material. More specifically, the drug-load may form a polymeric coating layer that completely or partially surrounds the core material wherein the drug-load comprises: (i) the drug; (ii) the binder; and (iii) optionally at least one additional pharmaceutical excipient.

The drug-loaded particles should have an average particle size less than about 1,500 μm, less than about 1,000 μm, less than about 850 μm, less than about 750 μm, or less than about 500 μm. In an embodiment of the present invention, drug-loaded particles should have an average particle size of about 1 μm to about 1000 μm, about 10 μm to about 900 μm, about 25 μm to about 800 μm, about 50 μm to about 750 μm or about 75 μm to about 500 μm.

The drug-loaded particles are formed into solid dosage forms by filing the drug-loaded particles into capsule shells or other suitable devices such as a sachet, pouch or vial or by combining the drug-loaded particles with at least one additional pharmaceutical excipient to create a final blend and filling the final blend into capsules or other suitable devices such as a sachet, pouch or vial or compressing the final blend into a tablet.

In certain embodiments of the present invention the core material, the drug-loading solution, suspension, or dispersion and/or the final blend comprising the drug-loaded particles may comprise additional pharmaceutical excipients. For example, the additional pharmaceutical excipients may comprise a filler, binder, lubricant, glidant, antioxidant, stabilizer, plasticizer, coloring agents, flavoring agents, or combinations thereof.

Suitable fillers for use in the present invention include, without limitation, microcrystalline cellulose, silicified microcrystalline cellulose, dibasic calcium phosphate, tribasic calcium phosphate, magnesium carbonate, magnesium oxide, calcium silicate, magnesium aluminum silicate, silicon dioxide, titanium dioxide, alumina, talc, kaolin, polyvinylpyrrolidone, dibasic calcium sulfate, tribasic calcium sulfate, starch, calcium carbonate, magnesium carbonate, carbohydrates, modified starches, lactose, sucrose, dextrose, mannitol, sorbitol, and inorganic compounds, or combinations thereof. In a preferred embodiment, the filler is colloidal silicon dioxide or microcrystalline cellulose. The filler may range from about 0.1% to about 70% by weight of the total weight of the final solid dosage form, or from about 0.5% to about 60% by weight of the total weight of the final solid dosage form or from about 1% to about 50% of the weight of the final solid dosage form. In one embodiment of the present invention the final blend includes the drug-loaded particles and a filler that is extra granular to the drug-loaded particles. In this embodiment, the amount of filler in the extra-granular component of the dosage forms of the present invention may range from about 5% to about 80% by weight of the extra-granular component, or about 5% to about 15%, or about 70% to about 80% by weight of the extra-granular component.

Suitable lubricants, glidants, or anti-adherent agents, also referred to as anti-caking agents, for use in the present invention include, without limitation, magnesium stearate, calcium stearate, stearic acid, sodium stearyl fumarate, hydrogenated vegetable oil (preferably comprised of hydrogenated and refined triglycerides of stearic and palmitic acids), tribasic calcium phosphate, calcium silicate, colloidal silicon dioxide, hydrophobic colloidal silica, magnesium oxide, magnesium silicate, magnesium trisilicate, silica, and talc, or combinations thereof. In one embodiment, the amount of anti-adherent agent in the drug-loading solution, suspension, or dispersion may range from about 0.05% to about 15% by weight of the total weight of the drug-loaded particle, preferably from about 0.1% to about 10% by weight of the total weight of the drug-loaded particle, and most preferably from about 0.5% to about 5% by weight of the total weight of the drug-loaded particle. In another embodiment, the amount of lubricants, glidants, or anti-adherent agents in the extra-granular portion of the final blend may be about 0.1% to about 15% by weight of the total weight of the final blend, preferably about 0.5% to about 10% of the total weight of the final blend.

Suitable antioxidants and stabilizers for use in the present invention include, without limitation, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate, or combinations thereof. In a preferred embodiment the antioxidant is citric acid monohydrate, butylated hydroxytoluene, or a combination thereof. In an embodiment of the present invention, the amount of antioxidant/stabilizer in the drug-loaded particles of the present invention may range from about 0% to about 5% by weight of the total weight of the drug-loaded particles, from about 0.01% to about 2.5% by weight of the total weight of the drug-loaded particles, or about 0.02% to about 2% by weight of the total weight of the drug-loaded particles. In another embodiment, the amount of antioxidant/stabilizer in the extra-granular component of the present invention may range from about 0% to about 20% by weight of the total weight of the final blend, from about 0.1% to about 15% by weight of the total weight of the final blend, or about 1% to about 10% by weight of the total weight of the final blend.

Suitable binders for use in the final blend of the present invention include, without limitation, starches (including corn starch and pregelatinized starch), gelatin, sugars (including sucrose, glucose, dextrose and lactose), polyethylene glycol, polyols, polyvinyl alcohols, C₁₂-C₁₈ fatty acid alcohols, waxes, gums (e.g., guar gum, arabic gum, acacia gum, xanthan gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, polymethacrylates, and polyvinyloxoazolidone, or combinations thereof. Preferred binders are water soluble materials, more preferably low molecule weight water soluble polymers and most preferably water soluble polymer that exhibit a viscosity of less than 75 cps when a 2% aqueous solution is prepared preferably less than 50 cps and most preferably less than 30 cps. The amount of binder in the extra-granular component or final blend of the present invention may range from about 0% to about 30% by weight of the total weight of the final blend, from about 0% to about 20% by weight of the total weight of the final blend, or about 0.5% to about 15% by weight of the total weight of the final blend.

Suitable plasticizers for use in the present invention include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons, which is incorporated in its entirety herein by reference. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and combinations thereof. In an embodiment of the present invention, the amount of plasticizer in the drug-loaded particles of the present invention may range from about 0% to about 15% by weight of the total weight of the drug-loaded particles, from about 0.01% to about 10% by weight of the total weight of the drug-loaded particles, or about 0.01% to about 5% by weight of the total weight of the drug-loaded particles.

Suitable coloring agents that may be employed in the present invention include pigments and dyes.

Suitable flavoring agents that may be employed in the present invention include artificial sweeteners such as aspartame, saccharin, dipotassium glycyrrhizinate, stevia, thaumatin, and flavorants such as citric acid, peppermint oil, wintergreen oil, menthol, lemon, lime, orange, grape, cherry, and vanilla extract. Additional taste enhancing agents are described in U.S. Pat. No. 6,027,746, which is incorporated in its entirety herein by reference.

In certain embodiments, the drug-loaded particles are optionally mixed with a lubricant or glidant, and loaded into a capsule to create a controlled release dosage form. More specifically, filling the drug-loaded particles into a capsule along with less than 20 wt %, preferably less than 10 wt % and most preferably less than 5 wt % of any additional excipients based on the total weight of the material inside the capsule shell will result in a controlled release solid dosage form when the capsule. Alternatively, the contents of the capsule shell may contain at least 80 wt % or more of the drug-loaded particles, at least 90 wt % or more of the drug-loaded particles, at least 95 wt % or more of the drug-loaded particles, at least 97 wt % or more of the drug-loaded particles, at least 99 wt % or more of the drug-loaded particles or 100 wt % of the drug-loaded particles. The rate of drug release may be modified or controlled by selecting the type, size and amount of the core material, the type and amount of binder for the drug-load and the amount of extra granular excipients within the capsule shell with the drug-loaded particles. For example, if about 10 wt % of a water soluble filler such as lactose is blended with the drug-loaded particles and the blend is filled into a capsule, the release rate of the drug from the capsule will be faster than the release rate of a capsule with a similar amount of drug-loaded particles but without the lactose because the lactose will deter or prevent the formation of a thick gel mass in situ.

It is believed that the in situ formation of a gel mass can contribute to abuse deterrent properties because the if an individual seeking to inject the drug product attempts to dissolve the solid dosage form in a small amount of water, the drug-loaded particles will hydrate creating a gel that is difficult to draw into a needle. Similarly, an individual seeking to snort the solid dosage form should be unable to obtain a quick dose of the drug because the drug-loaded particles will agglomerate and gel in the nasal region which may delay the release of the drug.

In certain embodiments of the present invention the solid dosage form comprises a final blend that comprises the drug-loaded particles and an extra-granular hydrophilic or gelling material and optionally at least one additional pharmaceutical excipient. The extra-granular hydrophilic or gelling material may be the same or different hydrophilic or gelling material as described above for the core material.

In an embodiment of the present invention, the amount of extra-granular hydrophilic or gelling polymer in the final blend may range from about 0.5% to about 15% by weight of the total weight of the final blend, preferably about 1% to about 10% by weight of the total weight of the final blend, and more preferably from about 1% to about 7.5% by weight of the total weight of the final blend.

Embodiments of the present invention comprise a plurality of the drug-loaded particles, optionally blended with the extra-granular components, and formed into a tablet or filled into capsules. In a preferred embodiment, the capsule is a hard shell capsule. Non-limiting examples of suitable hard shell capsules that may be used according to the present invention include hard starch capsules, hard gelatin capsules, hard cellulose capsules, and hydrogel capsules. Non-limiting types of tablets that may be used according to the present invention include coated tablets, uncoated tablets, multi-layer tablets. In a preferred embodiment, a plurality of drug-loaded particles and extra-granular material including at least one hydrophilic or gelling polymer and at least one additional pharmaceutical excipient are blended and compressed into tablets. In one embodiment of the present invention, the compressed tablets have a hardness from 50 N to 500 N, preferably 75 N to 400 N, and most preferably 100 N to 350 N when measured according to the method for determining the breaking strength of tablets published in the European Pharmacopoeia 1997, page 143-144, method no. 2.9.8.

The tablets may also be coated with a tablet coating that may be formulated for aesthetic purposes or for immediate release, extended release, or delayed release. Aesthetic coatings suitable for the present invention are sold under the trade name OPADRY by Colorcon. These coatings are available in a variety of colors as well as clear. In an embodiment, the tablet coating is sprayed onto the uncoated tablets until an about 1% to about 5% weight gain is achieved, preferably an about 3% weight gain.

The solid dosage forms of the present invention may provide extended release of the drug for up to about 6 hours, up to about 8 hours, up to about 10 hours, up to about 12 hours, up to about 16 hours, up to about 20 hours, or up to about 24 hours. In an embodiment of the present invention, the solid dosage forms provide a drug release when measured according to a USP Apparatus II (paddle) with sinker at 100 rpm in 900 mL of simulated intestinal fluid (without enzyme) at pH 6.8 (“dissolution media”) of about 20% to about 40% at 2 hours, about 40% to about 60% at 4 hours, about 50% to about 70% at 6 hours, about 60% to about 80% at 8 hours, about 70% to about 90% at 10 hours, about 80% to about 100% at 12 hours, and not less than 90% at 14 hours. In a preferred embodiment, the solid dosage forms provide a drug release when measured in dissolution media of about 25% to about 35% at 2 hours, about 40% to about 50% at 4 hours, about 55% to about 65% at 6 hours, about 70% to about 80% at 8 hours, about 75% to about 85% at 10 hours, about 85% to about 95% at 12 hours, and about 90% to about 100% at 13 hours. In another embodiment, the solid dosage forms provide a drug release when measured in dissolution media of about 30% to about 50% at 2 hours, about 50% to about 70% at 4 hours, about 60% to about 80% at 6 hours, about 70% to about 90% at 8 hours, about 80% to about 100% at 10 hours, and not less than 90% at 12 hours. In another preferred embodiment, the solid dosage forms provide a drug release when measured in dissolution media of about 35% to about 45% at 2 hours, about 55% to about 65% at 4 hours, about 70% to about 80% at 6 hours, about 80% to about 90% at 8 hours, about 85% to about 95% at 10 hours, and about 90% to about 100% at 12 hours. In a further embodiment, the solid dosage forms provide a drug release when measured in dissolution media of about 40% to about 60% at 2 hours, about 60% to about 80% at 4 hours, about 70% to about 90% at 6 hours, about 80% to about 100% at 8 hours, and not less than 90% at 10 hours. In a further preferred embodiment, the solid dosage forms provide a drug release when measured in dissolution media of about 40% to about 50% at 2 hours, about 65% to about 75% at 4 hours, about 80% to about 90% at 6 hours, about 85% to about 95% at 8 hours, and about 90% to about 100% at 10 hours.

The solid dosage forms of the present invention may also possess abuse-deterrent properties. In particular, the extended-release dosage forms are intended to impart abuse-deterrent properties in order to reduce the risk of improper administration of drugs when the dosage form is manipulated. In one embodiment, the hardness of the tablet of the present invention should be at a level that makes breaking the tablet by hand difficult. In certain embodiments the hardness should be at least 100 N or greater, at least 125 N or greater, at least 150 N of greater, at least 175 N or greater, at least 200 N or greater, at least 225 N or greater or at least 250 N or greater. Moreover, when a mechanical grinder is applied to the tablet, the result is particles of deformed drug-loaded granules that are either too large to be retained in the nasal cavities or that stick to the nasal membrane by forming a hydrogel immediately upon contact with the nasal membrane. The ground particles also resist solvent extraction for injection and/or ingestion due to the high concentration of hydrophilic polymer(s) in the composition. In addition, owing to the high concentration of low-melting point polymeric materials, it is difficult to heat the ground particles to abuse the drug by smoking as the individual particles start to melt and bind to each other to form a plastic mass when exposed to heat.

As a summary, the anti-abuse properties of the solid dosage forms of the present invention are listed in Table 1.

TABLE 1
Abuse Deterrent Properties of the Developed
Dosage Form Based on Main Abuse Routes.

| Abuse Routes | Abuse Deterrent Properties |
|---|---|
| Snorting | Hard tablet, not easy to break by hand; |
| | After mechanically manipulating a tablet or a capsule, the obtained |
| | large particles cannot stay in nasal cavities and/or pass through the |
| | nasal channel, while small particles stick to the nasal membranes as a |
| | hydrogel rapidly forms to reduce immediate release of the drug. |
| Injection | Difficult to extract the drug into small volume of water for injection |
| | due to hydrogel formation, even after grinding a tablet or taking out the |
| | contents from a capsule. |
| Ingestion | After mechanically manipulating a tablet or a capsule, the granulation |
| | still undergoes sustained release; |
| | When alcohol is taken with the administration of the drug, the dosage |
| | form presents a slower drug release rate than normal. |
| Smoking | Low melting point polymers undergo melting to form a plastic barrier |
| | to resist drug sublimation. |

A more detailed description of the various test methods for measuring the abuse deterrent properties of a dosage form can be found in U.S. Pat. No. 8,309,060 (break strength); U.S. Pat. No. 7,776,314 (needle extraction); and U.S. Pat. No. 9,084,816, which are incorporated in their entirety herein by reference.

It is believed that the hydrophilic or gelling polymer present in the core of the drug-loaded particles and the hydrophilic or gelling polymer present in the extra-granular portion of the final blend contribute to the abuse deterrent properties of the present invention. In certain embodiments, the solid dosage forms may also include additional abuse deterrent features. For example, in one embodiment, the dosage form may additionally comprise a bittering agent in the drug-loaded particle and/or in the extra-granular component to discourage an abuser from tampering with the dosage form and thereafter inhaling or swallowing the tampered dosage form. Preferably, the bittering agent is released when the dosage form is tampered with and provides an unpleasant taste to the abuser upon inhalation and/or swallowing of the tampered dosage form. Suitable bittering agents include natural, artificial, and synthetic flavor oils and flavoring aromatics and/or oils, oleoresins, and extracts derived from plants, leaves, flowers, fruits, and so forth, and combinations thereof. Non-limiting representative flavor oils include spearmint oil, peppermint oil, eucalyptus oil, oil of nutmeg, allspice, mace, oil of bitter almonds, menthol, and the like. Also useful bittering agents are artificial, natural, and synthetic fruit flavors such as citrus oils including lemon, orange, lime, grapefruit, and fruit essences and so forth. Additional bittering agents include sucrose derivatives (e.g., sucrose octaacetate), chlorosucrose derivatives, quinine sulphate, and the like. A further bittering agent for use in the present invention is Denatonium Benzoate NF-Anhydrous sold under the name BITREX (McFarlan Smith Limited, Edinburgh, UK). A bittering agent may be added to the formulation in an amount of less than about 50% by weight, preferably less than about 10% by weight, most preferably less than about 5% by weight of the dosage form, and most preferably in an amount ranging from about 0.1 to 1.0 percent by weight of the dosage form depending on the particular bittering agent(s) used.

In another embodiment, the solid dosage form additionally comprises an irritant to discourage an abuser from tampering with the solid dosage form and thereafter inhaling, injecting, or swallowing the tampered dosage form. Preferably, the irritant is released when the solid dosage form is tampered with and provides a burning or irritating effect to the abuser upon inhalation, injection, and/or swallowing the tampered dosage form. Suitable irritants include capsaicin, a capsaicin analog with similar type properties as capsaicin, and the like. Some capsaicin analogues or derivatives include for example and without limitation, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylamide, or other compounds of the class known as vanilloids. In a particular embodiment, capsaicin or analogues thereof are employed in a concentration between about 0.00125% and 50% by weight, preferably between about 1% and about 7.5% by weight, and most preferably between about 1% and about 5% by weight.

In another embodiment, the solid dosage form may additionally comprise an opioid antagonist such as naloxone, naltrexone, nalmefene, nalide, nalmexone, nalorphine, nalorphine dinicotinate, cyclazocine, levallorphan, and combinations thereof. The opioid antagonist may be present in the drug-loaded particle and/or in the extra-granular component to discourage an abuser from tampering with the solid dosage form and thereafter inhaling or swallowing the tampered dosage form. Preferably, the opioid antagonist is sequestered from the drug such as taught in U.S. Pat. No. 8,877,247 or 7,682,633, which are incorporated in their entirety herein by reference.

The present invention is also directed to a method for preparing the drug-loaded particles as described above and for preparing solid oral dosage forms, preferably a capsule or tablet that exhibits at least one of the abuse-deterrent features described in Table 1 above. In certain embodiments, the present invention is directed to a method for preparing a tablet that exhibits a break strength of at least 100 N, 125 N, 150 N, 175 N, 200 N, 225 N, 250 N, 275 N 300 N, 325 N, 350 N, 375 N, 400 N, 425 N, 450 N, 475 N, 500 N or higher without the need for a curing or heating step.

Embodiments of the present invention are further directed to a methods of treating disease or symptoms of disease such as pain, hypertension, arrhythmia, hyperlipidemia, hypoglycemia, seizures, infections, psychosis, anxiety, or stress by administering to a patient in need of such treatment a solid dosage form comprising a plurality of the drug-loaded particles described herein. In certain embodiments the drug employed in the solid dosage forms or the present invention and administered to a patient in need are water soluble drugs selected from the group of analgesic agents, anesthetic agents, anti-anginal agents, anti-anxiety agents, anticonvulsant agents, antidepressants, anti-inflammatory agents, antipsychotic agents, antitussive agents, appetite suppressant agents, relaxant agents, sedative-hypnotic agents, stimulants, tranquilizing agent and vasodilator agents as identified above.

Embodiments of the present invention is also directed to methods of treating pain in a patient by administering a solid dosage form described herein. In an embodiment the pain is acute pain or mild to moderate pain. In one embodiment the drug in the solid dosage form is an opioid, preferably tapentadol, oxymorphone, oxycodone, hydrocodone, morphine or a pharmaceutically acceptable salt thereof. If the solid dosage form is a capsule, the capsule may be opened and the contents sprinkled to a food product, such as applesauce or yogurt, and the food product with the capsule contents administered to a patient.

### EXAMPLES

### Example 1a

An extended-release tablet was prepared in accordance with the composition presented in Table 2 wherein metoprolol succinate was used as a model water soluble drug.

TABLE 2

| | | |
|---|---|---|
| Ingredient | mg/unit | % (w/w) |

Drug-Loading Dispersion

| | | |
|---|---|---|
| Metoprolol Succinate | 291.2 | 41.69 |
| Talc | 10.0 | 1.43 |
| Butylated Hydroxytoluene | 0.2 | 0.03 |
| EUDRAGIT NE 30 D (as solid) | 54.0 | 7.73 |
| FD&C Blue #1 Al Lake | 0.2 | 0.03 |
| Water (q.s.) | | |

Drug-Loaded Core

| | | |
|---|---|---|
| POLYOX WSR 303 | 210.0 | 30.06 |
| POLYOX WSR 1105 | 100.0 | 14.32 |

Extra-Granular

| | | |
|---|---|---|
| POLYOX WSR 303 | 13.6 | 1.95 |
| POLYOX WSR 1105 | 7.0 | 1.00 |
| Citric Acid Monohydrate | 3.5 | 0.50 |
| Colloidal Silicon Dioxide | 7.0 | 1.00 |
| Magnesium Stearate | 1.8 | 0.26 |
| Total | 698.5 | 100.00 |

### Preparation of Drug-Loading Dispersion

Metoprolol succinate was dissolved in purified water. Delumped talc and milled butylated hydroxytoluene were added under stirring. Followed by steadily adding EUDRAGIT NE 30 D into the solution with propeller stirring to make the drug-loading dispersion. FD&C Blue #1 A1 Lake was added to the drug-loading dispersion to aid in visulation of the drug-load.

### Preparation of Drug-Loaded Particles

POLYOX WSR 303 (polyethylene oxide, M.W. 7,000,000 Da) and POLYOX WSR 1105 (polyethylene oxide, M.W. 900,000 Da) with a particle size between 35-325 mesh was loaded into a Vector VFC-Lab 1 Fluid Bed Granulator using Wurster Technology after pre-heating. The powders were fluidized and the drug-loading dispersion was sprayed onto the powders when the temperature reached 30° C. The spray process continued until all the dispersion is sprayed. The drug-loaded particles were dried in the fluid bed granulator until the loss on drying (LOD) was not more than (NMT) 0.5%. The dried drug-loaded particles were discharge from the fluid bed granulator. The typical process parameters of drug-loading are presented in Table 3.

TABLE 3
Typical Process Parameters of Wurster Coating

| Parameters | Target (Range) | |
|---|---|---|
| Air flow (CFM) | 25 | (22-28) |
| Inlet temperature (° C.) | 50 | (45-52) |
| Product temperature (° C.) | 30 | (28-32) |
| Exhaust temperature (° C.) | 29 | (27-30) |
| Partition height (inch) | 0.75 | |
| Atomizing air (psi) | 25 | |
| Pattern air (psi) | 25 | |
| Nozzle diameter (mm) | 1.2 | |
| Spray rate (g/min) | 9 | (3-12) |

### Preparation of Final Blend

The drug-loaded particles were blended with extra-granular excipients including POLYOX WSR 303, POLYOX WSR 1105, citric acid monohydrate, and colloidal silicon dioxide. The final blend was achieved after a lubricating process in which magnesium stearate was added to the blend.

### Preparation of the Uncoated Tablet Core

The final blend was loaded into a rotary tablet press and compressed into tablets using 18.9 mm×8.1 mm oblong biconcave tooling sets. The target tablet weight is 700 mg, and the hardness of the tablet reaches 320 N after compression.

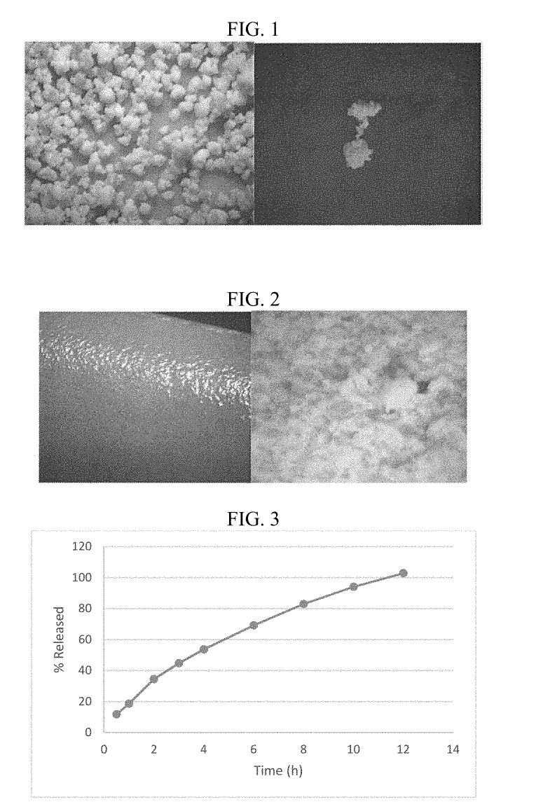

During the drug-loading process in the Wurster fluid bed granulator, the drug is applied onto the hydrophilic polymer core material (POLYOX WSR 303 and WSR 1105 having a particle size of about 60-125 mesh) with the water-insoluble polymeric binder (EUDRAGIT NE 30 D). After the drug-loading process, a drug-load was applied to the hydrophilic polymer core and forms a partial layer on the surface of polymer particles as seen in FIG. 1.

The drug-loaded particles may homogenously disperse in the final blend, and undergo plastic deformation under compression to bind to one another in the tablet. FIG. 2 shows the tablet surface composed of the drug-loaded particles, and the bound drug-loaded particles inside the tablet.

Drug Release from the Dosage Form

Dissolution testing was conducted to evaluate drug release performance under the parameters presented in Table 4.

TABLE 4
Dissolution Conditions

| | |
|---|---|
| Dosage Form | Tablet (Extended Release) |
| USP Apparatus | II (Paddle) with sinker |
| Speed | 100 RPM |
| Medium | 0.05M Phosphate Buffer of pH 6.8, Simulated |
| | intestinal fluid (without enzyme) |
| Volume | 900 mL |
| Sampling Times | 0.5, 1, 2, 3, 4, 6, 8, 10 and 12 hours |

Samples were analyzed by HPLC. The drug release profile is presented in FIG. 3 with the numerical values as follows.

| | Time (hr) | | | | | | | | |
|---|---|---|---|---|---|---|---|---|---|
| | 0.5 | 1 | 2 | 3 | 4 | 6 | 8 | 10 | 12 |
| % Released | 12 | 19 | 34 | 45 | 54 | 69 | 83 | 94 | 103 |

### Example 1b

Extended-release tablets may be prepared as described in Example 1a, with or without the FD&C dye, by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 2a

Extended-release tablets are prepared in accordance with the composition presented in Table 5 wherein metoprolol succinate was used as a model water soluble drug.

TABLE 5

| | | |
|---|---|---|
| Ingredient | mg/unit | % (w/w) |

Drug-Loading Dispersion

| | | |
|---|---|---|
| Metoprolol Succinate | 291.2 | 40.39 |
| Talc | 10.0 | 1.39 |
| EUDRAGIT NE 30 D | 54.0 | 7.49 |
| Water (q.s.) | | |

Drug-Loaded Core

| | | |
|---|---|---|
| POLYOX WSR 303 | 210.0 | 29.13 |
| POLYOX WSR 1105 | 100.0 | 13.87 |

Extra-Granular

| | | |
|---|---|---|
| POLYOX WSR 303 | 7.1 | 0.98 |
| POLYOX WSR 1105 | 3.0 | 0.42 |
| Citric Acid Monohydrate | 3.5 | 0.49 |
| Butylated Hydroxytoluene | 0.2 | 0.03 |
| Colloidal Silicon Dioxide | 14.0 | 1.94 |
| Sodium Stearyl Fumarate, PRUV | 7.0 | 0.97 |

Coated Tablet

| | | |
|---|---|---|
| OPADRY | 21.0 | 2.91 |
| Water (q.s.) | | |
| Total | 721.0 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make a drug-loading dispersion.

### Preparation of Drug-Loaded Particle

POLYOX WSR 303 and POLYOX WSR 1105 with a particle size between 35-325 mesh was loaded into a Vector VFC-Lab 1 Fluid Bed Granulator using Wurster Technology after pre-heating. The powders were fluidized. The drug-loading suspension was sprayed onto the powders once the powders reached a temperature of 30° C. The spraying continued until all the drug-loading dispersion was consumed. The drug-loaded particles were dried after spraying in the fluidized bed until the LOD was NMT 0.5% and discharged from the fluid bed. The typical process parameters of drug-loading are presented in Table 3.

### Preparation of Final Blend

The drug-loaded particles were blended with extra-granular excipients including POLYOX WSR 303, POLYOX WSR 1105, citric acid monohydrate, butylated hydroxytoluene, and colloidal silicon dioxide. The final blend was achieved after a lubricating process, in which sodium stearyl fumarate was added to the blend.

### Preparation of Uncoated Tablet Core

The final blend was loaded into a rotary tablet press and compressed into tablets using oblong biconcave tooling sets as described in Example 1a. The target tablet weight is 700 mg, and the hardness of the tablet reaches 320 N after compression.

### Preparation of Coated Tablet

An aesthetic coating was applied to the tablets using a Vector LDCS Hi-Coater equipped with a 15″ coating pan and a 1.2 mm nozzle spray gun. OPADRY was sprayed onto the uncoated tablet cores until 3% target weight gain was achieved. The process parameters are presented in Table 6.

TABLE 6
Typical Process Parameters of Tablet Coating

| Parameters | Target (Range) | |
|---|---|---|
| Air Volume (CFM) | 60 | (55-62) |
| Inlet air temperature (° C.) | 60 | (55-62) |
| Exhaust temperature (° C.) | 42 | (40-45) |
| Atomizing air (psi) | 20 | |
| Pattern air (psi) | 20 | |
| Pan speed (rpm) | 12 | (10-14) |
| Spray rate (g/min) | 8 | (6-10) |

Drug Release from the Dosage Form

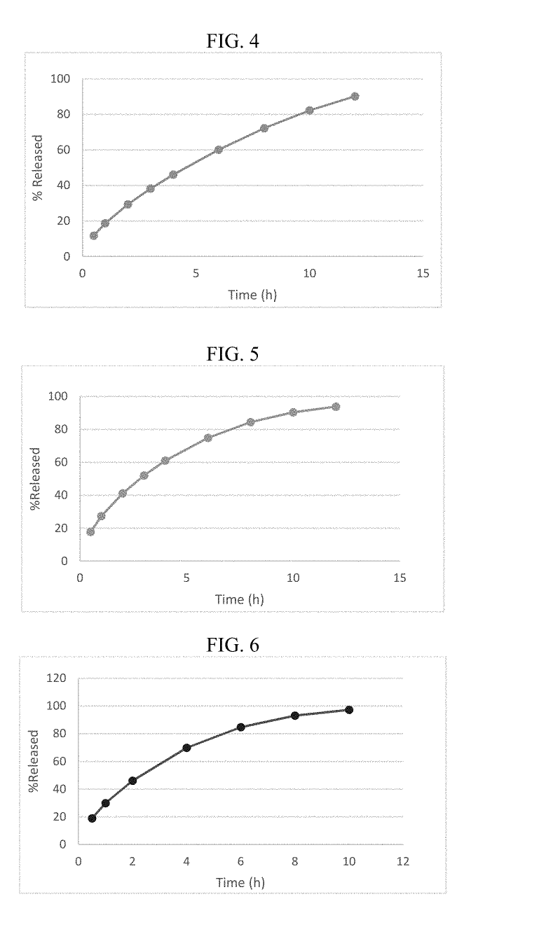

Dissolution testing was conducted to evaluate drug release performance under the parameters presented in Table 4. Samples were analyzed by HPLC. The drug release profile is presented in FIG. 4 with the numerical values as follows.

| | Time (hr) | | | | | | | | |
|---|---|---|---|---|---|---|---|---|---|
| | 0.5 | 1 | 2 | 3 | 4 | 6 | 8 | 10 | 12 |
| % Released | 12 | 19 | 29 | 38 | 46 | 60 | 72 | 82 | 90 |

### Example 2b

Extended-release tablets may be prepared as described in Example 2a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 3a

An extended-release capsule was prepared in accordance with the composition presented in Table 7 wherein metoprolol succinate was used as a model water soluble drug.

TABLE 7

| | | |
|---|---|---|
| Ingredient | mg/unit | % (w/w) |

Drug-Loading Dispersion

| | | |
|---|---|---|
| Metoprolol succinate | 291.2 | 43.76 |
| Talc | 10.0 | 1.50 |
| EUDRAGIT NE 30 D | 54.0 | 8.12 |
| FD&C Al Lake Dye (optional) | 0.2 | 0.03 |
| Water (q.s.) | | |

Drug-Loaded Core

| | | |
|---|---|---|
| POLYOX WSR 303 | 210.0 | 31.56 |
| POLYOX WSR 1105 | 100.0 | 15.03 |
| Total | 665.4 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make the drug-loading dispersion.

### Preparation of Drug-Loaded Particles

POLYOX WSR 303 and POLYOX WSR 1105 with a particle size between 35 and 325 mesh was loaded into a Vector VFC-Lab 1 Fluid Bed Granulator using Wurster Technology after pre-heating. The powders were fluidized. The drug-loading dispersion was sprayed onto the powders when the temperature reached to 30° C. Spraying continued until the drug-loading dispersion was consumed. After spraying the drug-loaded particles were dried in the fluidized bed to until LOD was NMT 0.5% and discharged from the fluid bed granulator. The typical process parameters of drug-loading are presented in Table 3.

### Encapsulation of Drug-Loaded Particles

The drug-loaded particles were encapsulated in a hard shell capsule.

Drug Release from the Dosage Form

Dissolution testing was conducted to evaluate drug release performance under the parameters presented in Table 4. Samples were analyzed by HPLC. The drug release profile is presented in FIG. 5 with the numerical values as follows.

| | Time (hr) | | | | | | | | |
|---|---|---|---|---|---|---|---|---|---|
| | 0.5 | 1 | 2 | 3 | 4 | 6 | 8 | 10 | 12 |
| % Released | 18 | 27 | 41 | 52 | 61 | 75 | 84 | 90 | 94 |

When the content of a capsule was exposed to dissolution media upon the breakage of the capsule shell, the granulation did not leak out and spread throughout the fluid but rather stuck together to form a loose matrix surrounded by a fully hydrated gel layer caused by the hydrophilic polymers.

### Example 3b

Extended-release capsules may be prepared as described in Example 3a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 4a

Extended-release tablets were prepared in accordance with the composition presented in Table 8 wherein metoprolol succinate was used as a model water soluble drug.

TABLE 8

| | | |
|---|---|---|
| Ingredient | mg/unit | % (w/w) |

Drug-Loading Dispersion

| | | |
|---|---|---|
| Metoprolol succinate | 40.00 | 19.05 |
| Talc | 2.00 | 0.95 |
| EUDRAGIT NE 30 D (as solid) | 15.00 | 7.14 |
| Water (q.s.) | | |

Drug-Loaded Core

| | | |
|---|---|---|
| POLYOX WSR 303 | 58.00 | 27.62 |
| POLYOX WSR 1105 | 28.00 | 13.33 |

Extra-Granular

| | | |
|---|---|---|
| POLYOX WSR 303 | 6.00 | 2.86 |
| POLYOX WSR 1105 | 3.00 | 1.43 |
| Butylated Hydroxytoluene | 0.20 | 0.10 |
| Citric Acid Monohydrate | 5.30 | 2.52 |
| PROSOLV SMCC HD 90 | 52.00 | 24.76 |
| Magnesium Stearate | 0.50 | 0.24 |
| Total | 210.00 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make the drug-loading dispersion.

### Preparation of Drug-Loaded Particles

POLYOX WSR 303 and POLYOX WSR 1105 with a particle size between 35 and 325 mesh was loaded into a Vector VFC-Lab 1 Fluid Bed Granulator using Wurster Technology after pre-heating. The powders were fluidized. The drug-loading dispersion was sprayed onto the powder when the temperature reached 30° C. The spraying continued until the drug-loading dispersion was consumed. After spraying the drug-loaded particles were dried in the fluidized bed until the LOD was NMT 0.5% and discharge from the fluid bed. The typical process parameters of drug-loading are presented in Table 3.

### Preparation of Final Blend

The drug-loaded particles were blended with extra-granular excipients including POLYOX WSR 303, POLYOX WSR 1105, citric acid monohydrate, butylated hydroxytoluene, and silicified microcrystalline cellulose (PROSOLV SMCC HD 90). The final blend was achieved after a lubricating process, in which magnesium stearate is added to the blend.

### Preparation of Tablet Core

The final blend was loaded into a rotary tablet press and compressed into tablets using 8.8 mm round biconvex tooling sets. The target tablet weight is 210 mg, and the hardness of the tablet reaches 100 N after compression.

Drug Release from the Dosage Form

Dissolution testing was conducted on an Agilent online UV dissolution system to evaluate drug release performance under the parameters presented in Table 9.

TABLE 9
Dissolution Conditions

| | |
|---|---|
| Dosage Form | Tablet (Extended Release) |
| USP Apparatus | II (Paddle) with sinker |
| Speed | 50 RPM |
| Medium | 45 mM Phosphate Buffer, pH 4.5 |
| Volume | 900 mL |
| Sampling Times | 1, 2, 4, 6, 8, and 10 hours |

The drug release profile is presented in FIG. 6 with the numerical values as follows.

| | Time (hr) | | | | | | |
|---|---|---|---|---|---|---|---|
| | 0.5 | 1 | 2 | 4 | 6 | 8 | 10 |
| % Released | 19 | 30 | 46 | 70 | 85 | 93 | 97 |

### Example 4b

Extended-release capsules may be prepared as described in Example 4a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 5

An extended-release, abuse-deterrent tapentadol HCl ER tablets, 250 mg was prepared in accordance with the composition presented in Table 10.

TABLE 10

| | | |
|---|---|---|
| Ingredient | mg/unit | % (w/w) |

Drug-Loading Dispersion

| | | |
|---|---|---|
| Tapentadol HCl | 291.2 | 41.69 |
| Talc | 10.0 | 1.43 |
| EUDRAGIT NE 30 D (as solid) | 54.0 | 7.73 |
| Water (q.s.) | | |

Drug-Loaded Core

| | | |
|---|---|---|
| POLYOX WSR 303 | 310.0 | 30.06 |

Extra-Granular

| | | |
|---|---|---|
| POLYOX WSR 303 | 27.8 | 1.95 |
| Citric Acid Monohydrate | 3.5 | 0.50 |
| Colloidal Silicon Dioxide | 3.5 | 1.00 |
| Total | 698.5 | 100.00 |

### Preparation of Drug-Loading Dispersion

Tapentadol HCl was dissolved in purified water. Delumped talc was added under stirring. Followed by steadily adding EUDRAGIT NE 30 D into the solution with propeller stirring to make the drug-loading dispersion.

### Preparation of Drug-Loaded Particles

POLYOX WSR 303 (polyethylene oxide, M.W. 7,000,000 Da) with a particle size between 35-325 mesh was loaded into a Fluid Bed Granulator using Wurster Technology after pre-heating. The powders were fluidized. The drug-loading dispersion was sprayed onto the powders when the temperature reached 30° C. The spraying continued until the drug-loading dispersion was consumed. After spraying the drug-loaded particles were dried in the fluidized bed until LOD was NMT 0.5% and discharged from the fluid bed. The typical process parameters of drug-loading are presented in Table 3.

### Preparation of Final Blend

The drug-loaded particles were blended with extra-granular excipients including POLYOX WSR 303, citric acid monohydrate, and colloidal silicon dioxide.

### Preparation of Tablet

The final blend was loaded into a rotary tablet press and compressed into tablets using oblong biconcave tooling sets.

Drug Release from the Dosage Form

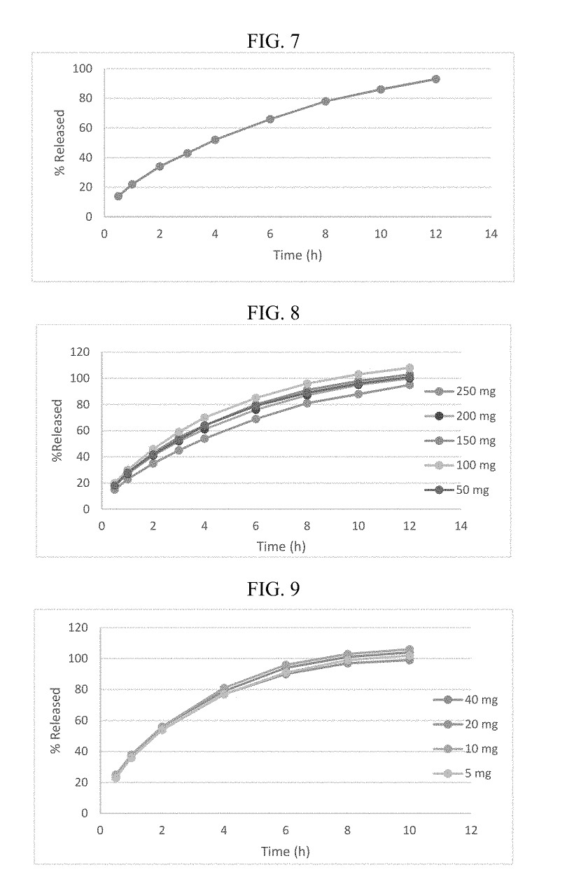

Dissolution testing was conducted to evaluate drug release performance under the parameters presented in Table 4. Samples were analyzed by HPLC. The drug release profile is presented in FIG. 7 with the numerical values as follows.

| | Time (hr) | | | | | | | | |
|---|---|---|---|---|---|---|---|---|---|
| | 0.5 | 1 | 2 | 3 | 4 | 6 | 8 | 10 | 12 |
| % Released | 14 | 22 | 34 | 43 | 52 | 66 | 78 | 86 | 93 |

### Example 6

The extended-release, abuse-deterrent tapentadol HCl ER tablets were prepared in accordance with the composition presented in Table 11.

TABLE 11

| | Strength | | | | | | | | | |
|---|---|---|---|---|---|---|---|---|---|---|
| | 250 mg | | 200 mg | | 150 mg | | 100 mg | | 50 mg | |
| Ingredient | mg/unit | % (w/w) | mg/tab | %, w/w | mg/tab | %, w/w | mg/tab | %, w/w | mg/tab | %, w/w |

Drug-loading Dispersion

| | | | | | | | | | | |
|---|---|---|---|---|---|---|---|---|---|---|
| Tapentadol HCl | 291.20 | 40.39 | 232.96 | 40.39 | 174.72 | 37.70 | 116.48 | 28.27 | 58.24 | 14.14 |
| Talc | 10.00 | 1.39 | 8.00 | 1.39 | 6.40 | 1.38 | 6.00 | 1.46 | 6.00 | 1.46 |
| EUDRAGIT NE 30 D (as | 54.00 | 7.49 | 43.20 | 7.49 | 34.80 | 7.51 | 31.20 | 7.57 | 31.20 | 7.57 |
| solid) | | | | | | | | | | |
| Water (q.s.) | | | | | | | | | | |

Drug-Loaded Core

| | | | | | | | | | | |
|---|---|---|---|---|---|---|---|---|---|---|
| POLYOX WSR 303 | 210.00 | 29.13 | 168.00 | 29.13 | 168.00 | 36.25 | 160.00 | 38.83 | 160.00 | 38.83 |
| POLYOX WSR 1105 | 100.00 | 13.87 | 80.00 | 13.87 | 32.00 | 6.90 | 52.00 | 12.62 | 52.00 | 12.62 |

Extra-Granular

| | | | | | | | | | | |
|---|---|---|---|---|---|---|---|---|---|---|
| POLYOX WSR 303 | 7.10 | 0.98 | 5.68 | 0.98 | 12.13 | 2.62 | 14.22 | 3.45 | 50.46 | 12.25 |
| POLYOX WSR 1105 | 3.00 | 0.42 | 2.40 | 0.42 | 6.00 | 1.29 | 6.00 | 1.46 | 28.00 | 6.80 |
| Citric Acid Monohydrate | 3.50 | 0.49 | 2.80 | 0.49 | 2.30 | 0.50 | 2.00 | 0.49 | 2.00 | 0.49 |
| Butylated hydroxytoluene | 0.20 | 0.03 | 0.16 | 0.03 | 0.15 | 0.03 | 0.10 | 0.02 | 0.10 | 0.02 |
| Colloidal Silicon Dioxide | 14.00 | 1.94 | 11.20 | 1.94 | 9.00 | 1.94 | 8.00 | 1.94 | 8.00 | 1.94 |
| Sodium Stearyl Fumarate | 7.00 | 0.97 | 5.60 | 0.97 | 4.50 | 0.97 | 4.00 | 0.97 | 4.00 | 0.97 |
| Subtotal | 700.00 | 97.09 | 560.00 | 97.09 | 450.00 | 97.09 | 400.00 | 97.09 | 400.00 | 97.09 |

Coated Tablet

| | | | | | | | | | | |
|---|---|---|---|---|---|---|---|---|---|---|
| OPADRY | 21.00 | 2.91 | 16.80 | 2.91 | 13.50 | 2.91 | 12.00 | 2.91 | 12.00 | 2.91 |
| Water (q.s.) | | | | | | | | | | |
| Total | 721.00 | 100.00 | 576.80 | 100.00 | 463.50 | 100.00 | 412.00 | 100.00 | 412.00 | 100.00 |

The tablets were prepared according to the procedure described in Example 2.

Dissolution testing was conducted to evaluate drug release performance under the parameters presented in Table 4. Samples were analyzed by HPLC. The drug release profile is presented in FIG. 8 with the numerical values as follows.

| | Time (hr) | | | | | | | | |
|---|---|---|---|---|---|---|---|---|---|
| | 0.5 | 1 | 2 | 3 | 4 | 6 | 8 | 10 | 12 |
| 250 mg | 15 | 23 | 35 | 45 | 54 | 69 | 81 | 88 | 95 |
| 200 mg | 18 | 27 | 41 | 52 | 61 | 76 | 87 | 95 | 100 |
| 150 mg | 19 | 28 | 43 | 55 | 64 | 80 | 91 | 98 | 103 |
| 100 mg | 20 | 30 | 46 | 59 | 70 | 85 | 96 | 103 | 108 |
| 50 mg | 18 | 28 | 42 | 53 | 64 | 79 | 89 | 96 | 101 |

### Example 7

Extended-release, abuse-deterrent Oxymorphone HCl tablets were prepared in accordance with the composition presented in Table 12.

TABLE 12

| | Strength | | | | | | | |
|---|---|---|---|---|---|---|---|---|
| | 40 mg | | 20 mg | | 10 mg | | 5 mg | |
| Ingredient | mg/unit | % (w/w) | mg/unit | % (w/w) | mg/unit | % (w/w) | mg/unit | % (w/w) |

Drug-Loading Dispersion

| | | | | | | | | |
|---|---|---|---|---|---|---|---|---|
| Oxymorphone HCl | 40.00 | 20.00 | 20.00 | 10.03 | 10.00 | 5.10 | 5.00 | 2.53 |
| Talc | 2.00 | 1.00 | 1.00 | 0.50 | 0.50 | 0.25 | 0.25 | 0.13 |
| EUDRAGIT NE 30 D (as solid) | 15.00 | 7.50 | 7.50 | 3.76 | 3.75 | 1.91 | 1.88 | 0.95 |
| Water (q.s.) | | | | | | | | |

Drug-Loaded Core

| | | | | | | | | |
|---|---|---|---|---|---|---|---|---|
| POLYOX WSR 303 | 58.00 | 29.00 | 29.00 | 14.54 | 10.00 | 5.10 | 7.25 | 3.66 |
| POLYOX WSR 1105 | 28.00 | 14.00 | 14.00 | 7.02 | 10.00 | 5.10 | 3.50 | 1.77 |

Extra-Granular

| | | | | | | | | |
|---|---|---|---|---|---|---|---|---|
| POLYOX WSR 303 | 3.00 | 1.50 | 30.00 | 15.04 | 45.00 | 22.93 | 50.00 | 25.27 |
| POLYOX WSR 1105 | 3.00 | 1.50 | 18.00 | 9.02 | 32.00 | 16.31 | 32.00 | 16.17 |
| Citric Acid Monohydrate | 5.00 | 2.50 | 5.00 | 2.51 | 5.00 | 2.55 | 5.00 | 2.53 |
| Lactose Monohydrate | 15.00 | 7.50 | 40.00 | 20.05 | 45.00 | 22.93 | 50.00 | 25.27 |
| Microcrystalline Cellulose, | 28.00 | 14.00 | 32.00 | 16.04 | 32.00 | 16.31 | 40.00 | 20.21 |
| AVICEL PH102 | | | | | | | | |
| Colloidal Silicon Dioxide | 2.00 | 1.00 | 2.00 | 1.00 | 2.00 | 1.02 | 2.00 | 1.01 |
| Sodium Stearyl Fumarate, PRUV | 1.00 | 0.50 | 1.00 | 0.50 | 1.00 | 0.51 | 1.00 | 0.51 |
| Total | 200.00 | 100.00 | 199.50 | 100.00 | 196.25 | 100.00 | 197.88 | 100.00 |

The tablets were prepared according to the procedure described in Example 1.

Dissolution testing was conducted to evaluate drug release performance under the parameters presented in Table 9. Samples were analyzed by HPLC. The drug release profiles and data are presented in FIG. 9 with the numerical values as follows.

| | Time (hr) | | | | | | |
|---|---|---|---|---|---|---|---|
| | 0.5 | 1 | 2 | 4 | 6 | 8 | 10 |
| 40 mg | 24 | 37 | 56 | 79 | 94 | 101 | 104 |
| 20 mg | 23 | 36 | 54 | 77 | 90 | 97 | 99 |
| 10 mg | 25 | 38 | 56 | 81 | 96 | 103 | 106 |
| 5 mg | 23 | 36 | 54 | 77 | 91 | 99 | 102 |

### Example 8a

An extended-release tablet was prepared in accordance with the composition presented in Table 13, wherein metoprolol succinate was used as a model water soluble drug.

TABLE 13

| | | |
|---|---|---|
| Ingredient | mg/unit | % (w/w) |

Drug-Loading Dispersion

| | | |
|---|---|---|
| Metoprolol Succinate | 40.00 | 19.42 |
| Talc | 2.00 | 0.97 |
| EUDRAGIT NE 30 D (as solid) | 15.00 | 7.28 |
| Water (q.s.) | | |

Drug-Loaded Core

| | | |
|---|---|---|
| POLYOX WSR 303 | 58.00 | 28.16 |
| POLYOX WSR 1105 | 28.00 | 13.59 |

Extra-Granular

| | | |
|---|---|---|
| POLYOX WSR 303 | 3.00 | 1.46 |
| POLYOX WSR 1105 | 3.00 | 1.46 |
| Citric Acid Monohydrate | 5.00 | 2.43 |
| Silicified Microcrystalline Cellulose | 45.00 | 21.84 |
| Sodium Stearyl Fumarate, PRUV | 1.00 | 0.49 |

Coated Tablet

| | | |
|---|---|---|
| OPADRY II | 6.00 | 2.91 |
| Water (q.s.) | | |
| Total | 206.00 | 100.00 |

The tablets were prepared according to the procedure described in Example 2.

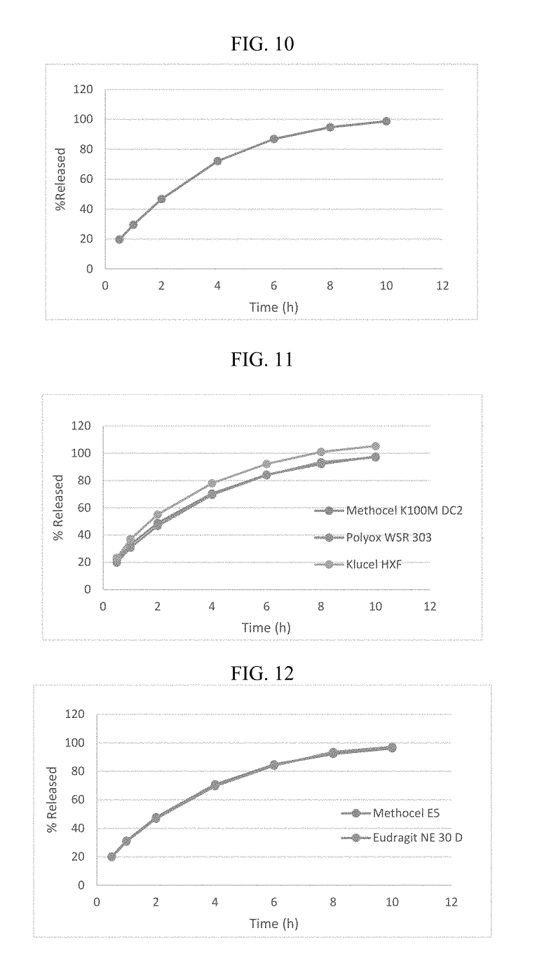

Dissolution testing was conducted on an Agilent online UV dissolution system to evaluate drug release performance from the tablets under the parameters presented in Table 9. The drug release profiles and data are presented in FIG. 10 and numerically as follows:

| | Time (hr) | | | | | | |
|---|---|---|---|---|---|---|---|
| | 0.5 | 1 | 2 | 4 | 6 | 8 | 10 |
| 40 mg | 20 | 30 | 47 | 72 | 87 | 95 | 99 |

### Example 8b

Extended-release capsules may be prepared as described in Example 8a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 9a

Extended-release tablets were prepared in accordance with the composition presented in Table 14 wherein metoprolol succinate was used as a model water soluble drug.

TABLE 14

| | Formulation 1 | | Formulation 2 | | Formulation 3 | |
|---|---|---|---|---|---|---|
| | | % | | % | | % |
| Ingredient | mg/unit | (w/w) | mg/unit | (w/w) | mg/unit | (w/w) |

Drug Loading Dispersion

| | | | | | | |
|---|---|---|---|---|---|---|
| Metoprolol Succinate | 40.00 | 18.60 | 40.00 | 18.60 | 40.00 | 18.58 |
| Talc | 2.00 | 0.93 | 2.00 | 0.93 | 2.00 | 0.93 |
| EUDRAGIT NE 30 D (as solid) | 15.00 | 6.98 | 15.00 | 6.98 | 15.00 | 6.97 |
| Water (q.s.) | | | | | | |

Drug Loaded Core

| | | | | | | |
|---|---|---|---|---|---|---|
| POLYOX WSR 303 | 68.00 | 31.63 | 0.00 | 0.00 | 0.00 | 0.00 |
| METHOCEL K100M DC2 | 0.00 | 0.00 | 68.00 | 31.63 | 0.00 | 0.00 |
| KLUCEL HXF | 0.00 | 0.00 | 0.00 | 0.00 | 68.00 | 31.58 |
| Colloidal Silicon Dioxide | 0.00 | 0.00 | 0.00 | 0.00 | 0.34 | 0.16 |

Extra-Granular

| | | | | | | |
|---|---|---|---|---|---|---|
| POLYOX WSR 303 | 6.00 | 2.79 | 0.00 | 0.00 | 0.00 | 0.00 |
| METHOCEL K100M DC2 | 0.00 | 0.00 | 6.00 | 2.79 | 0.00 | 0.00 |
| KLUCEL HXF | 0.00 | 0.00 | 0.00 | 0.00 | 6.00 | 2.79 |
| Citric Acid Monohydrate | 5.00 | 2.33 | 5.00 | 2.33 | 5.00 | 2.32 |
| Lactose Monohydrate | 15.00 | 6.98 | 15.00 | 6.98 | 15.00 | 6.97 |
| Microcrystalline Cellulose, | | | | | | |
| AVICEL PH102 | 59.60 | 27.72 | 59.60 | 27.72 | 59.60 | 27.68 |
| Colloidal Silicon Dioxide | 2.20 | 1.02 | 2.20 | 1.02 | 2.20 | 1.02 |
| Sodium Stearyl Fumarate, | | | | | | |
| PRUV | 2.20 | 1.02 | 2.20 | 1.02 | 2.20 | 1.02 |
| Total | 215.00 | 100.00 | 215.00 | 100.00 | 215.34 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make a drug-loading dispersion.

### Preparation of Drug-Loaded Particle

POLYOX WSR 303, METHOCEL K100M DC2 and KLUCEL HXF for Formulations 1, 2, and 3, respectively, with a particle size between 35-325 mesh were loaded into a Fluid Bed Granulator using Wurster Technology after pre-heating. Colloidal silicon dioxide was added for Formulation 3 to improve flowability. The powders were fluidized. The drug-loading dispersion was sprayed onto the powders when the temperature reached 30° C. The spraying process continue until all the drug-loading dispersion was consumed. After spraying the drug-loaded particles were dried in the fluidized bed until LOD was NMT 0.5% and discharged from the fluid bed granulator. The typical process parameters of drug-loading are presented in Table 3.

### Preparation of Final Blend

The drug-loaded particles were blended with extra-granular excipients including POLYOX WSR 303 for Formulation 1, METHOCEL K100M DC2 for Formulation 2, KLUCEL HXF for Formulation 3, citric acid monohydrate, lactose monohydrate, microcrystalline cellulose, and colloidal silicon dioxide. The final blend was achieved after a lubricating process, in which sodium stearyl fumarate is added to the blend.

### Preparation of Tablet

The final blend was loaded into a rotary tablet press and compressed into tablets using round biconvex tooling sets.

Drug Release from the Dosage Form

Dissolution testing is conducted to evaluate drug release performance under the parameters presented in Table 9. The drug release profiles and data are presented in FIG. 11 and numerically as follows.

| | Time (hr) | | | | | | |
|---|---|---|---|---|---|---|---|
| | 0.5 | 1 | 2 | 4 | 6 | 8 | 10 |
| POLYOX WSR 303 | 20 | 31 | 47 | 70 | 84 | 94 | 97 |
| METHOCEL K100M DC2 | 22 | 33 | 49 | 70 | 84 | 92 | 98 |
| KLUCEL HXF | 23 | 37 | 55 | 78 | 92 | 101 | 105 |

### Example 9b

Extended-release capsules may be prepared as described in Example 9a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 10a

Extended-release tablets were prepared in accordance with the composition presented in Table 15, wherein metoprolol succinate is used as a model water soluble drug.

TABLE 15

| | Formulation 1 | | Formulation 2 | |
|---|---|---|---|---|
| Ingredient | mg/unit | % (w/w) | mg/unit | % (w/w) |

Drug-Loading dispersion

| | | | | |
|---|---|---|---|---|
| Metoprolol Succinate | 40.00 | 18.60 | 40.00 | 18.60 |
| Talc | 2.00 | 0.93 | 2.00 | 0.93 |
| METHOCEL E5 | 7.50 | 3.49 | 0.00 | 0.00 |
| EUDRAGIT NE 30 D | 0.00 | 0.00 | 15.00 | 6.98 |
| (as solid) | | | | |
| Water (q.s.) | | | | |

Drug-Loaded Core

| | | | | |
|---|---|---|---|---|
| POLYOX WSR 303 | 68.00 | 31.63 | 68.00 | 31.63 |

Extra-Granular

| | | | | |
|---|---|---|---|---|
| POLYOX WSR 303 | 6.00 | 2.79 | 6.00 | 2.79 |
| Citric Acid Monohydrate | 5.00 | 2.33 | 5.00 | 2.33 |
| Lactose Monohydrate | 15.00 | 6.98 | 15.00 | 6.98 |
| Microcrystalline Cellulose, | 67.10 | 31.21 | 59.60 | 27.72 |
| AVICEL PH102 | | | | |
| Colloidal Silicon Dioxide | 2.20 | 1.02 | 2.20 | 1.02 |
| Sodium Stearyl Fumarate, | 2.20 | 1.02 | 2.20 | 1.02 |
| PRUV | | | | |
| Total | 215.00 | 100.00 | 215.00 | 100.00 |

### Preparation of Drug-Loading Dispersion

Metoprolol succinate was dissolved in purified water. Delumped talc was added under stirring. Followed by steadily adding binder, METHOCEL E5 and EUDRAGIT NE 30D, for Formulations 1, and 2, respectively, into the solution with propeller stirring to make a drug-loading dispersion.

### Preparation of Drug-Loaded Particle

POLYOX WSR 303 with a particle size between 35-325 mesh was loaded into a Fluid Bed Granulator using Wurster Technology after pre-heating. The powders were fluidized. The drug-loading dispersion was sprayed onto the powders and continued until the drug-loading dispersion was consumed. After spraying, the drug-loaded particles were dried in the fluidized bed until the LOD was NMT 0.5% and discharged from the fluid bed granulator. The typical process parameters of drug-loading are presented in Table 3.

### Preparation of Final Blend

The drug-loaded particles were blended with extra-granular excipients including POLYOX WSR 303, citric acid monohydrate, lactose monohydrate, microcrystalline cellulose, and colloidal silicon dioxide. The final blend was achieved after a lubricating process, in which sodium stearyl fumarate is added to the blend.

### Preparation of Tablet

The final blend was loaded into a rotary tablet press and compressed into tablets using 8.8 mm round biconvex tooling sets.

Drug Release from the Dosage Form

Dissolution testing was conducted to evaluate drug release performance under the parameters presented in Table 9. The drug release profiles are presented in FIG. 12 and numerically as follows:

| | Time (hr) | | | | | | |
|---|---|---|---|---|---|---|---|
| | 0.5 | 1 | 2 | 4 | 6 | 8 | 10 |
| METHOCEL E5 | 20 | 31 | 48 | 71 | 85 | 92 | 96 |
| EUDRAGIT NE 30 D | 20 | 31 | 47 | 70 | 84 | 94 | 97 |

### Example 10b

Extended-release capsules may be prepared as described in Example 10a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 11a

Extended-release tablets were prepared in accordance with the composition presented in Table 16, wherein metoprolol succinate is used as a model water soluble drug.

TABLE 16

| | | |
|---|---|---|
| Ingredient | mg/unit | % (w/w) |

Drug-Loading Dispersion

| | | |
|---|---|---|
| Metoprolol Succinate | 116.48 | 29.12 |
| Talc | 6.00 | 1.50 |
| EUDRAGIT NE 30 D (as solid) | 31.20 | 7.80 |
| Water (q.s.) | | |

Drug-Loaded Core

| | | |
|---|---|---|
| POLYOX WSR 1105 | 226.00 | 56.50 |

Extra-Granular

| | | |
|---|---|---|
| POLYOX WSR 303 | 4.22 | 1.06 |
| POLYOX WSR 1105 | 2.00 | 0.50 |
| Citric Acid Monohydrate | 2.00 | 0.50 |
| Butylated Hydroxytoluene | 0.10 | 0.03 |
| Colloidal Silicon Dioxide | 8.00 | 2.00 |
| Sodium Stearyl Fumarate | 4.00 | 1.00 |
| Total | 400.00 | 100.00 |

The tablets were prepared according to the procedure described in Example 1.

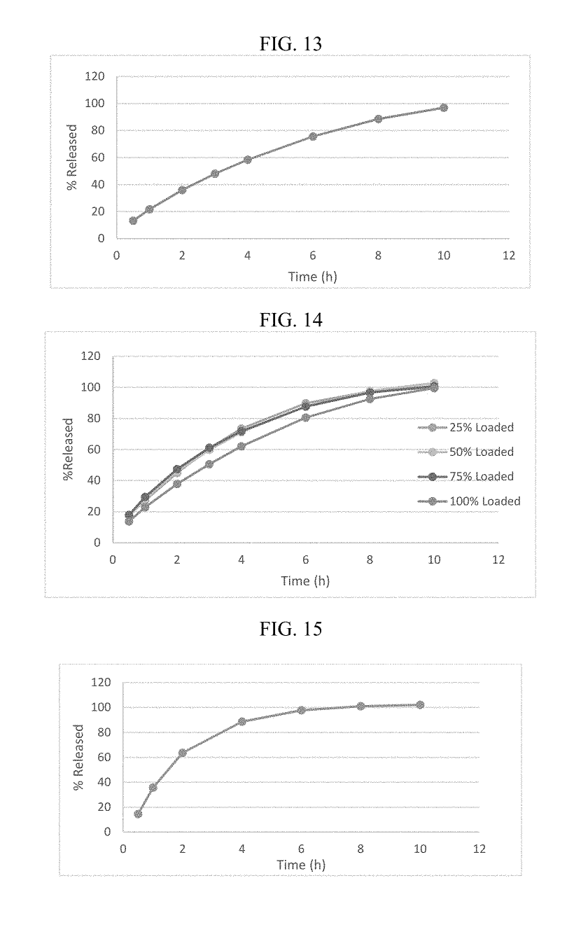

Dissolution testing was conducted to evaluate drug release performance from the tablets under the parameters presented in Table 4. The drug release profiles are presented in FIG. 13 and numerically as follows.

| | Time (hr) | | | | | | | |
|---|---|---|---|---|---|---|---|---|
| | 0.5 | 1 | 2 | 3 | 4 | 6 | 8 | 10 |
| % Released | 13 | 22 | 36 | 48 | 58 | 76 | 89 | 97 |

### Example 11b

Extended-release capsules may be prepared as described in Example 11a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 12a

Extended-release tablets were prepared in accordance with the composition presented in Table 17, wherein metoprolol succinate was used as a model water soluble drug.

TABLE 17

| | Loading Level | | | | | | | |
|---|---|---|---|---|---|---|---|---|
| | 25% Loading | | 50% Loading | | 75% Loading | | 100% Loading | |
| Ingredient | mg/unit | % (w/w) | mg/unit | % (w/w) | mg/unit | % (w/w) | mg/unit | % (w/w) |

Drug-Loading Dispersion

| | | | | | | | | |
|---|---|---|---|---|---|---|---|---|
| Metoprolol Succinate | 29.12 | 11.01 | 58.24 | 19.23 | 87.36 | 25.60 | 116.48 | 30.68 |
| Talc | 1.50 | 0.57 | 3.00 | 0.99 | 4.50 | 1.32 | 6.00 | 1.58 |
| EUDRAGIT NE 30 D (as solid) | 7.80 | 2.95 | 15.60 | 5.15 | 23.40 | 6.86 | 31.20 | 8.22 |
| Water (q.s.) | | | | | | | | |

Drug-Loaded Core

| | | | | | | | | |
|---|---|---|---|---|---|---|---|---|
| POLYOX WSR 1105 | 226.00 | 85.47 | 226.00 | 74.63 | 226.00 | 66.23 | 226.00 | 59.52 |
| Total | 264.42 | 100.00 | 302.84 | 100.00 | 341.26 | 100.00 | 379.68 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make a drug-loading dispersion.

### Preparation of Drug-Loaded Particle

POLYOX WSR 1105 with a particle size between 35-325 mesh was loaded into a Fluid Bed Granulator using Wurster Technology after pre-heating. The powders were fluidized. The drug-loading dispersion was sprayed onto the powders when the temperature reached 30° C. The spraying process continued until the drug-loading dispersion was consumed. After spraying, the drug-loaded particles were dried in the fluidized bed until the LOD was NMT 0.5% and discharged from the fluid bed granulator. The typical process parameters of drug-loading are presented in Table 3.

### Preparation of Tablet

The drug-loaded granulation was loaded into a rotary tablet press and compressed into tablets using round biconcave tooling sets.

Drug Release from the Dosage Form

Dissolution testing was conducted to evaluate drug release performance under the parameters presented in Table 4. The drug release profiles are presented in FIG. 14 and numerically as follows.

| | Time (hr) | | | | | | | |
|---|---|---|---|---|---|---|---|---|
| | 0.5 | 1 | 2 | 3 | 4 | 6 | 8 | 10 |
| 25% Loaded | 18 | 27 | 45 | 61 | 73 | 90 | 97 | 100 |
| 50% Loaded | 17 | 28 | 46 | 60 | 71 | 88 | 98 | 103 |
| 75% Loaded | 18 | 30 | 48 | 61 | 72 | 88 | 97 | 101 |
| 100% Loaded | 14 | 23 | 38 | 51 | 62 | 81 | 93 | 100 |

### Example 12b

Extended-release capsules may be prepared as described in Example 12a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 13a

Extended-release capsules were prepared in accordance with the composition presented in Table 18 wherein metoprolol succinate was used as a model water soluble drug.

TABLE 18

| | | |
|---|---|---|
| Ingredient | mg/unit | % (w/w) |

Drug-Loading Dispersion

| | | |
|---|---|---|
| Metoprolol Succinate | 40.00 | 34.04 |
| Talc | 2.00 | 1.70 |
| METHOCEL E5 | 7.50 | 6.38 |
| Water (q.s.) | | |

Drug-Loaded Core

| | | |
|---|---|---|
| POLYOX WSR 303 | 68.00 | 57.87 |
| Total | 117.50 | 100.00 |

### Preparation of Drug-Loading Dispersion

Metoprolol succinate was dissolved in purified water. METHOCEL E5 was steadily added into the solution with propeller stirring followed by delumped talc under stirring to make the drug-loading dispersion.

### Preparation of Drug-Loaded Particles

POLYOX WSR 303 with a particle size between 35 and 325 mesh was loaded into a Vector VFC-Lab 1 Fluid Bed Granulator using Wurster Technology after pre-heating. The powder was fluidized. The drug-loading dispersion was sprayed onto the powders when the temperature reached 30° C. The spraying process continued until the drug-loading dispersion was consumed. After spraying, the drug-loaded particles were dried in the fluidized bed until the LOD was NMT 0.5% and discharged from the fluid bed granulator. The typical process parameters of drug-loading are presented in Table 3.

### Encapsulation of Drug-Loaded Particles

The drug-loaded particles were encapsulated in an HMPC hard shell capsule.

Drug Release from the Dosage Form

Dissolution testing was conducted on an Agilent online UV dissolution system to evaluate drug release performance under the parameters presented in Table 9. The drug release profile is presented in FIG. 15 and numerically as follows.

| | Time (hr) | | | | | | |
|---|---|---|---|---|---|---|---|
| | 0.5 | 1 | 2 | 4 | 6 | 8 | 10 |
| % Released | 14 | 36 | 64 | 89 | 98 | 101 | 102 |

### Example 13b

Extended-release capsules may be prepared as described in Example 13a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 14a

Extended-release capsules were prepared in accordance with the composition presented in Table 19 wherein metoprolol succinate was used as a model water soluble drug.

TABLE 19

| | | |
|---|---|---|
| Ingredient | mg/unit | % (w/w) |

Drug-Loading Dispersion

| | | |
|---|---|---|
| Metoprolol Succinate | 40.00 | 27.97 |
| Talc | 2.00 | 1.40 |
| EUDRAGIT NE 30 D (as solid) | 15.00 | 10.49 |
| Water (q.s.) | | |

Drug-Loaded Core

| | | |
|---|---|---|
| POLYOX WSR 303 | 58.00 | 40.56 |
| POLYOX WSR 303 | 28.00 | 19.58 |
| Total | 143.00 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make a drug-loading dispersion.

### Preparation of Drug-Loaded Particles

POLYOX WSR 303 and PILYOX WSR 1105 with a particle size between 35 and 325 mesh was loaded into a Vector VFC-Lab 1 Fluid Bed Granulator using Wurster Technology after pre-heating. The powders were fluidized. The drug-loading dispersion was sprayed onto the powders when the temperature reached 30° C. The spraying process continued until the drug-loading dispersion was consumed. After spraying, the drug-loaded particles were dried in the fluidized bed until the LOD was NMT 0.5% and discharged from the fluid bed granulator. The typical process parameters of drug-loading are presented in Table 3.

### Encapsulation of Drug-Loaded Particles

The drug-loaded particles were encapsulated in an HMPC hard shell capsule.

Drug Release from the Dosage Form

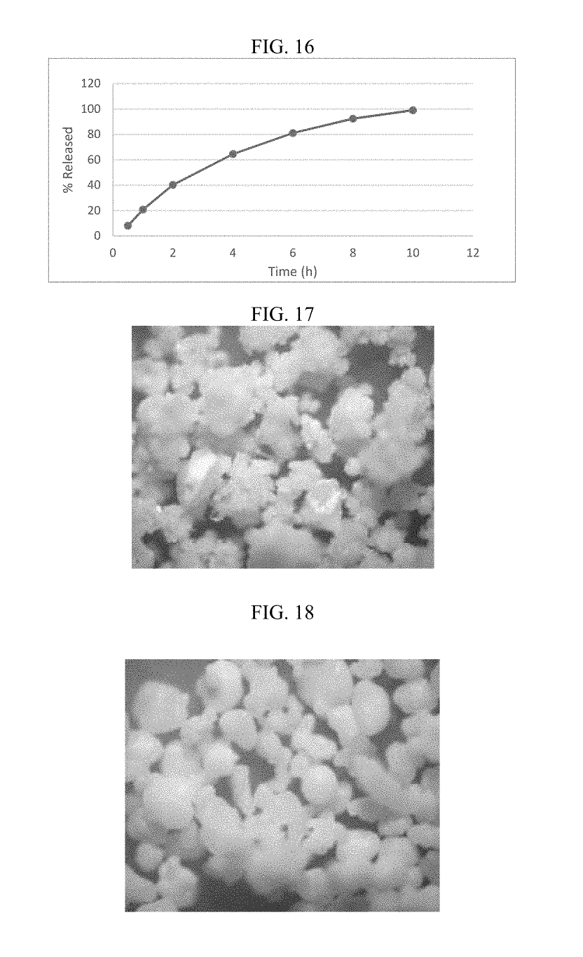

Dissolution testing was conducted on an Agilent online UV dissolution system to evaluate drug release performance under the parameters presented in Table 9. The drug release profile is presented in FIG. 16 and numerically as follows.

| | Time (hr) | | | | | | |
|---|---|---|---|---|---|---|---|
| | 0.5 | 1 | 2 | 4 | 6 | 8 | 10 |
| % Released | 8 | 21 | 40 | 65 | 81 | 93 | 99 |

### Example 14b

Extended-release capsules may be prepared as described in Example 14a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 15

The following extended-release solid dosage forms are prepared by direct compression, high shear granulation and drug-loading techniques wherein metoprolol succinate was used as a model water soluble drug.

Composition 15a [Direct Compression]—

Metoprolol Succinate ER Tablets prepared by dry blending the ingredients in the following table and compressing the blend into a tablet.

| | | |
|---|---|---|
| Ingredient | mg/unit | % (w/w) |

Blend

| | | |
|---|---|---|
| Metoprolol Succinate | 291.20 | 41.60 |
| POLYOX WSR 303 | 237.60 | 33.94 |
| POLYOX WSR 1105 | 100.00 | 14.29 |
| EUDRAGIT L100-55 | 54.00 | 7.71 |
| Talc | 10.00 | 1.43 |
| Citric Acid Monohydrate | 3.50 | 0.50 |
| Butylated Hydroxytoluene | 0.20 | 0.03 |
| Magnesium Stearate | 3.50 | 0.50 |
| Total | 700.00 | 100.00 |

Composition 15b [High Shear Granulation]—

Metoprolol Succinate ER Tablets prepared with the following composition by wet granulating the granular material with ethanol in a Vector GMXB-LAB Micro High-Shear Granulator equipped with a 4 L bowl, blending the resulting granules with the extra-granular components and compressing the blend into a tablet.

| | | |
|---|---|---|
| Ingredient | mg/unit | % (w/w) |

Granulation

| | | |
|---|---|---|
| Metoprolol Succinate | 291.20 | 41.60 |
| POLYOX WSR 303 | 210.00 | 30.00 |
| POLYOX WSR 1105 | 100.00 | 14.29 |
| EUDRAGIT L100-55 | 54.00 | 7.71 |
| Ethanol (q.s.) | | |

Extra-Granular

| | | |
|---|---|---|
| POLYOX WSR 303 | 27.60 | 3.94 |
| Talc | 10.00 | 1.43 |
| Citric Acid Monohydrate | 3.50 | 0.50 |
| Butylated Hydroxytoluene | 0.20 | 0.03 |
| Magnesium Stearate | 3.50 | 0.50 |
| Total | 700.00 | 100.00 |

The following parameters were applied to the high shear granulation process.

| | |
|---|---|
| Parameters | Target |

Pre-Blend

| | |
|---|---|
| Impeller Speed (rpm) | 720 |
| Time (min) | 3 |

Infusion and Wet Mass

| | |
|---|---|
| Impeller Speed (rpm) | 500 |
| Chopper Speed (rpm) | 1000 |
| Pump Speed (rpm) | 90 |
| Time (min) | 2 |

Composition 15c [Fluid Bed Drug Loading]—

Metoprolol Succinate ER Tablet prepared with the following composition according to the procedure described in Example 1a.

| | | |
|---|---|---|
| Ingredient | mg/unit | % (w/w) |

Drug-loading Dispersion

| | | |
|---|---|---|
| Metoprolol Succinate | 291.20 | 41.60 |
| Talc | 10.00 | 1.43 |
| Butylated Hydroxytoluene | 0.20 | 0.03 |
| EUDRAGIT NE 30 D (as solid) | 54.00 | 7.71 |
| Water (q.s.) | | |

Drug-Loaded Core

| | | |
|---|---|---|
| POLYOX WSR 303 | 210.00 | 30.00 |
| POLYOX WSR 1105 | 100.00 | 14.29 |

Extra-Granular

| | | |
|---|---|---|
| POLYOX WSR 303 | 27.60 | 3.94 |
| Citric Acid Monohydrate | 3.50 | 0.50 |
| Colloidal Silicon Dioxide | 3.50 | 0.50 |
| Total | 700.00 | 100.00 |

### Physical Appearance of Granulation/Particles:

The physical appearance of granulation/particles are visually observed and compared under stereo-microscopy at the same conditions. The figures are captured and presented in FIGS. 17 and 18 for granules from composition 15b (high shear granulator), and the granules from composition 15c (fluid bed drug loading) respectively.

Tablets as described in 15a, 15b and 15c were prepared by compressing the final blend of powders into a tablet using an 18.9 mm×8.1 mm oblong biconcave tooling set with a target weight of 700 mg.

Capsule samples employing the uncompressed powders with the composition described in 15a, 15b and 15c were prepared by encapsulating the final blend of powders (half dose) into size 0 hard shell capsule with a target weight of 350 mg.

Dissolution testing is conducted on the tablets and capsules to evaluate the drug release under the conditions presented in Table 20.

TABLE 20

| | |
|---|---|
| USP Apparatus | II (Paddle) with sinker |
| Speed | 100 RPM |
| Medium | 0.05M Phosphate Buffer of pH 6.8, |
| | Simulated intestinal fluid |
| | (without enzyme) |
| Volume | 900 mL |
| Sampling Times | 0.5, 1, 2, 3, 4, 6, 8, 10 and 12 hours |

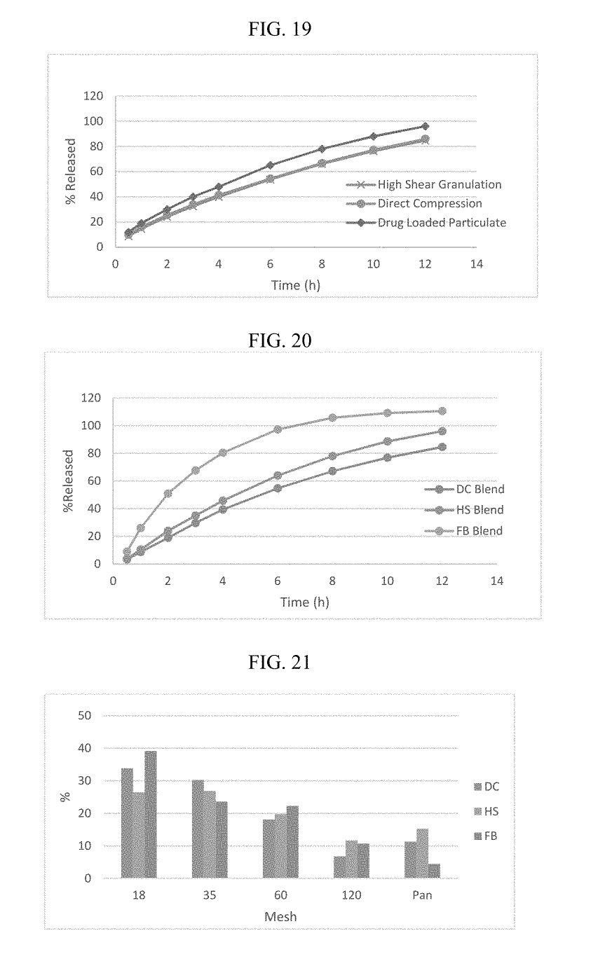

The tablet samples were analyzed by HPLC. The drug release profiles from the tablets are presented in FIG. 19 and numerically as follows:

| | Time (hr) | | | | | | | | |
|---|---|---|---|---|---|---|---|---|---|
| | 0.5 | 1 | 2 | 3 | 4 | 6 | 8 | 10 | 12 |
| Direct Compression | 10 | 16 | 26 | 34 | 41 | 55 | 67 | 77 | 86 |
| High Shear Granulation | 9 | 15 | 24 | 32 | 40 | 54 | 66 | 76 | 85 |
| Drug Loaded Particulate | 12 | 19 | 30 | 40 | 48 | 65 | 78 | 88 | 96 |

The capsule samples were analyzed by on-line UV detector. The drug release profiles from the capsules are presented in FIG. 20 and numerically as follows:

| | Time (hr) | | | | | | | | |
|---|---|---|---|---|---|---|---|---|---|
| | 0.5 | 1 | 2 | 3 | 4 | 6 | 8 | 10 | 12 |
| Direct Compression | 3 | 9 | 19 | 30 | 39 | 55 | 67 | 77 | 85 |
| High Shear Granulation | 4 | 10 | 24 | 35 | 46 | 64 | 78 | 89 | 96 |
| Drug Loaded Particulate | 9 | 26 | 51 | 68 | 80 | 97 | 106 | 109 | 110 |

The abuse deterrent properties of the tablets described in 15 a, 15b and 15c were evaluated. In the first evaluation, three tablets as described in 15a, 15b and 15c were placed into a grinder, IKA A10 Basic Grinder with A10.1 Stainless Steel Cutter and subjected to three (3) 10 second grinding pulses. After the 3 grinding pulses, the contents were transferred to a sonic sifter for sieve testing and the particle size distribution is shown in Table 21 and FIG. 21.

TABLE 21
Particle Size Distribution of Ground Tablets
from different processing methods

| | mesh | | | | |
|---|---|---|---|---|---|
| Sample | 18 | 35 | 60 | 120 | Pan |
| Direct Compression | 39.2 | 23.6 | 22.2 | 10.7 | 4.4 |
| High Shear Granulation | 33.8 | 30.2 | 18.0 | 6.8 | 11.3 |
| Drug Loaded Particulate | 26.4 | 26.9 | 19.7 | 11.7 | 15.2 |

The data in the Table 21 and FIG. 21 show that tablets prepared in accordance with the present invention exhibit a particle size that should make snorting a tampered tablet difficult.

In a second evaluation, one tablet as described in 15a, 15b and 15c were placed in an IKA A10 Basic grinder with A10.1 Stainless Steel Cutter as described above and subjected to three (3) 10 second grinding pulses. After the three pulses, the contents were transferred to a 20-mL glass bottle. 5 mL and 10 mL aliquots of water were separately added to each bottle and the contents gently swirl for a few seconds then allowed to rest for 2 minutes. A syringe (BD Disposable Syringe with Luer-Lok Tip, 5 mL and 10 mL) with a needle (BD Precisionglide™ hyperdermic conventional needle 20G×1.5 inch [0.9 mm×40 mm]) to withdraw the liquids into a 50-mL centrifuge tube. The withdrawn volumes are shown in Table 22.

TABLE 22
The Volumes that the syringe withdrawn
from Different Sample Preparations

Volumes withdrawn (mL)

| Samples | 5 mL | 10 mL |
|---|---|---|
| Direct Compression | 0 | 5 |
| High Shear Granulation | 0 | 5.2 |
| Drug Loaded Particulate | 0 | 5.5 |

An additional 45 mL of water was added into the fluid drawn into the 50-mL centrifuge tubes from the 10 mL extraction and the centrifuge tubes were capped and gently invert 30 times. After the inversion the tubes were centrifuge at 3500 rpm for 7 minutes. After centrifuging the supernatants were filtered through a 0.45 um syringe w/GHP Acrodisc membranes. The first 2 mL of the filtrate were discarded and about 3 mL of filtrate were collected. 1 mL of the collected filtrate was transferred into a 10-mL volumetric flask, dilute to volume with water and mixed well for HPLC analysis. The results are shown in Table 23.

TABLE 23
The percentage of extracted Drug from
10 mL Withdrawn Samples from Table 22

| Samples | Percentage of extracted API from tablet (%) |
|---|---|
| Direct Compression | 18.03 |
| High Shear Granulation | 20.91 |
| Drug Loaded Particulate | 17.70 |

The data in the Tables 22 and 23 show that tablets prepared in accordance with the present invention exhibit a viscosity and release properties that should make IV abuse of a tampered tablet difficult.

### Example 16

An extended-release formulation was prepared in accordance with the composition presented in Table 24, where metoprolol succinate was used as a model drug to form tablets and

### Preparation of Polymeric Core

POLYOX WSR 303 and POLYOX WSR 1105 with a particle size between 35-325 mesh was loaded into a Vector GMXB-LAB Micro High-Shear Granulator equipped with a 4 L bowl. The polymers were pre-blended followed by infusing the 90% ethanol to the power particles to the ending point of granulation. The granulation was discharged, passed through an 18 mesh sieve, and loaded into a Vector VFC-Lab 1 Fluid Bed Dryer. The polymeric particles were dried in the fluidized bed until the LOD was NMT 0.5%; discharged from the fluid bed; then passed through a 12 mesh sieve. Typical process parameters of high shear granulation are presented in Table 25.

TABLE 25
Typical Process Parameters of High Shear Granulation

| | |
|---|---|
| Parameters | Target |

Pre-Blend

| | |
|---|---|
| Impeller Speed (rpm) | 720 |
| Time (min) | 3 |

Infusion and Wet Mass

| | |
|---|---|
| Impeller Speed (rpm) | 720 |
| Chopper Speed (rpm) | 1000 |
| Pump Speed (rpm) | 90 |
| Time (min) | 3 |

### Preparation of Drug-Loading Dispersion

Metoprolol succinate was dissolved in purified water. Milled butylated hydroxytoluene and delumped talc were added under stirring. Followed by steadily adding EUDRAGIT NE 30 D into the solution with propeller stirring to make a drug-loading dispersion. FD&C Blue #1 A1 Lake was added to the drug-loading dispersion to aid in visualization of the drug load.

### Preparation of Drug-Loaded Particle

The polymeric cores were loaded into a Vector VFC-Lab 1 Fluid Bed Granulator using Wurster Technology after pre-heating. The polymeric cores were fluidized. The drug-loading dispersion was sprayed onto the polymeric cores when the temperature reached 30° C. The spraying process continued until the drug-loading dispersion was consumed. After the spraying process, the drug-loaded particles were dried until the LOD was NMT 0.5% and discharged from the fluid bed. The typical process parameters of drug-loading were presented in Table 3.

### Preparation of Final Blend

The drug-loaded particles were blended with extra-granular excipients including POLYOX WSR 303, POLYOX WSR 1105, citric acid monohydrate, and colloidal silicon dioxide. The final blend was achieved after a lubricating process, in which magnesium stearate was added to the blend.

### Preparation of Tablet

The final blend was loaded into a rotary tablet press and compressed into tablets using 18.9 mm×8.1 mm oblong biconcave tooling sets.

### Preparation of Capsule

The final blend is encapsulated in an HMPC hard shell “00” capsule.

Drug Release from the Dosage Form

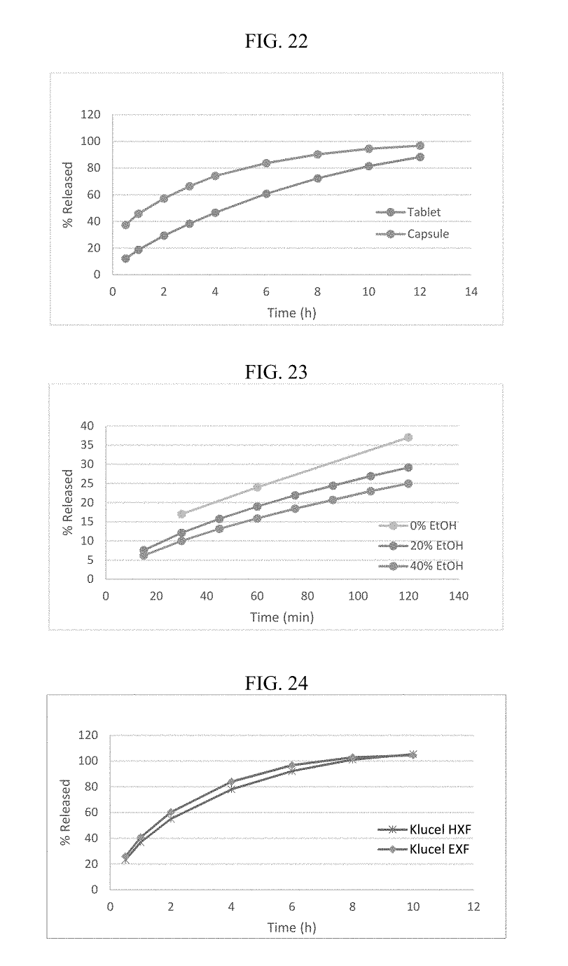

Dissolution testing was conducted to evaluate drug release performance under the parameters presented in Table 4. Samples were analyzed by HPLC. The drug release profile and data are presented in FIG. 22 and numerically as follows.

| | Time (hr) | | | | | | | | |
|---|---|---|---|---|---|---|---|---|---|
| | 0.5 | 1 | 2 | 3 | 4 | 6 | 8 | 10 | 12 |
| Tablet | 12 | 19 | 29 | 38 | 47 | 61 | 72 | 81 | 88 |
| Capsule | 37 | 46 | 57 | 66 | 74 | 84 | 90 | 94 | 97 |

### Example 17

An extended-release tablet is prepared in accordance with the composition presented in Table 26 wherein metoprolol succinate was used as a model water soluble drug.

TABLE 26

| | | |
|---|---|---|
| Ingredient | mg/unit | % (w/w) |

Drug-Loading Dispersion

| | | |
|---|---|---|
| Metoprolol Succinate | 291.2 | 41.65 |
| Talc | 10.0 | 1.43 |
| EUDRAGIT NE 30 D | 54.0 | 7.72 |
| FD&C Blue #1 Al Lake | 0.20 | 0.03 |
| Water (q.s.) | | |

Drug-Loaded Core

| | | |
|---|---|---|
| POLYOX WSR 303 | 210.0 | 30.04 |
| POLYOX WSR 1105 | 100.0 | 14.30 |

Extra-Granular

| | | |
|---|---|---|
| POLYOX WSR 303 | 16.10 | 2.30 |
| POLYOX WSR 1105 | 5.10 | 0.73 |
| Butylated hydroxytoluene | 0.20 | 0.03 |
| Citric acid Monohydrate | 3.50 | 0.50 |
| Colloidal Silicon Dioxide | 7.00 | 1.00 |
| Magnesium Stearate | 1.80 | 0.26 |
| Total | 699.10 | 100.00 |

### Preparation of Drug-Loading Dispersion

Metoprolol succinate was dissolved in purified water. Delumped talc was added under stirring. Followed by steadily adding EUDRAGIT NE 30 D into the solution with propeller stirring to make a drug-loading dispersion.

### Preparation of Drug-Loaded Particle

POLYOX WSR 303 and POLYOX WSR 1105 with a particle size between 35-325 mesh was loaded into a Vector VFC-Lab 1 Fluid Bed Granulator using Wurster Technology after pre-heating. The powders were fluidized. The drug-loading suspension was sprayed onto the powders once the powders reached a temperature of 30° C. The spraying continued until all the drug-loading dispersion was consumed. The drug-loaded particles were dried after spraying in the fluidized bed until the LOD was NMT 0.5% and discharged from the fluid bed. The typical process parameters of drug-loading are presented in Table 3.

### Preparation of Final Blend

The drug-loaded particles were blended with extra-granular excipients including POLYOX WSR 303, POLYOX WSR 1105, citric acid monohydrate, butylated hydroxytoluene, and colloidal silicon dioxide. The final blend was achieved after a lubricating process, in which sodium stearyl fumarate was added to the blend.

### Preparation of Uncoated Tablet Core

The final blend was loaded into a rotary tablet press and compressed into tablets using oblong biconcave tooling sets as described in Example 1a.

Drug Release from the Dosage Form

Dissolution testing was conducted in 0.1 N HCl media with ethanol at 0%, 20%, and 40%, respectively, to evaluate drug release performance under the parameters presented in Table 27.

TABLE 27
Dissolution Conditions

| | |
|---|---|
| Dosage Form | Tablet (Extended Release) |
| USP Apparatus | II (Paddle) with sinker |
| Speed | 100 RPM |
| Medium | 0.1 N HCl with Ethanol at 0%, 20%, and 40% |
| Volume | 900 mL |

Samples were analyzed by HPLC. The drug release profile is presented in FIG. 23 with the numerical values as follows.

| | Time (min) | | | | | | | |
|---|---|---|---|---|---|---|---|---|
| | 15 | 30 | 45 | 60 | 75 | 90 | 105 | 120 |
| 0% EtOH | — | 17 | — | 24 | — | — | — | 37 |
| 20% EtOH | 8 | 12 | 16 | 19 | 22 | 24 | 27 | 29 |
| 40% EtOH | 6 | 10 | 13 | 16 | 18 | 21 | 23 | 25 |

### Example 18a

The extended-release tablets were prepared in accordance with the compositions presented in Table 28, wherein metoprolol succinate is used as a model water soluble drug.

TABLE 28
Formulations of Metoprolol Succinate ER Tablets

| | Formulation 1 | | Formulation 2 | |
|---|---|---|---|---|
| Ingredient | mg/unit | % (w/w) | mg/unit | % (w/w) |

Dispersion Drug Loading

| | | | | |
|---|---|---|---|---|
| Metoprolol Succinate | 40.00 | 18.58 | 40.00 | 18.55 |
| Talc | 2.00 | 0.93 | 2.00 | 0.93 |
| EUDRAGIT NE 30 D | 15.00 | 6.97 | 15.00 | 6.95 |
| (as solid) | | | | |
| Water (q.s.) | | | | |

Drug Loaded Core

| | | | | |
|---|---|---|---|---|
| Hydroxypropyl Cellulose, | 68.00 | 31.58 | 0.00 | 0.00 |
| KLUCEL HXF* | | | | |
| Hydroxypropyl Cellulose, | 0.00 | 0.00 | 68.00 | 31.53 |
| KLUCEL EXF** | | | | |
| Colloidal Silicon Dioxide | 0.34 | 0.16 | 0.68 | 0.32 |

Extra-Granular

| | | | | |
|---|---|---|---|---|
| Hydroxypropyl Cellulose, | 6.00 | 2.79 | 0.00 | 0.00 |
| KLUCEL HXF | | | | |
| Hydroxypropyl Cellulose, | 0.00 | 0.00 | 6.00 | 2.78 |
| KLUCEL EXF | | | | |
| Citric Acid Monohydrate | 5.00 | 2.32 | 5.00 | 2.32 |
| Lactose Monohydrate | 15.00 | 6.97 | 15.00 | 6.95 |
| Microcrystalline Cellulose, | 59.60 | 27.68 | 59.60 | 27.63 |
| AVICEL PH102 | | | | |
| Colloidal Silicon Dioxide | 2.20 | 1.02 | 2.20 | 1.02 |
| Sodium Stearyl Fumarate, | 2.20 | 1.02 | 2.20 | 1.02 |
| PRUV | | | | |
| Total | 215.34 | 100.00 | 215.68 | 100.00 |

*KLUCEL HXF has a molecular weight of approximately 1,150 kDa.
**KLUCEL EXF has a molecular weight of approximately 80 kDa.

The drug-loaded particles and final blend were prepared according to the procedure described in Example 1a. The final blend was loaded into a rotary tablet press and compressed into tablets using 8.8 mm round biconvex tooling sets. Dissolution testing was conducted on an Agilent online UV dissolution system to evaluate drug release performance under the parameters presented in Table 4. The drug release profiles are presented in FIG. 24 with the numerical values as follows.

| | Time (hr) | | | | | | |
|---|---|---|---|---|---|---|---|
| | 0.5 | 1 | 2 | 4 | 6 | 8 | 10 |
| Formulation 1 | 23 | 36 | 55 | 77 | 92 | 101 | 105 |
| Formulation 2 | 26 | 41 | 60 | 84 | 97 | 103 | 104 |

### Example 18b

Extended-release capsules may be prepared as described in Example 18a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

### Example 19a

The extended-release capsules were prepared in accordance with the compositions presented in Table 29, wherein metoprolol succinate is used as a model water soluble drug.

The drug-loaded particles were prepared according to the procedure described in Example 1a.

### Preparation of Final Blend

The drug-loaded particles were blended with the extra-granular excipients including POLYOX WSR 303 for Formulation 2 only, citric acid monohydrate, lactose monohydrate, microcrystalline cellulose, and colloidal silicon dioxide. The final blend was achieved after a lubricating process, in which sodium stearyl fumarate was added to the blend.

### Encapsulation of Final Blend

Approximately 215 mg of the final blend was encapsulated in a size 00 HMPC hard shell capsule.

Drug Release from the Capsule

Dissolution testing is conducted on the capsules to evaluate the drug release under the conditions presented in Table 9.

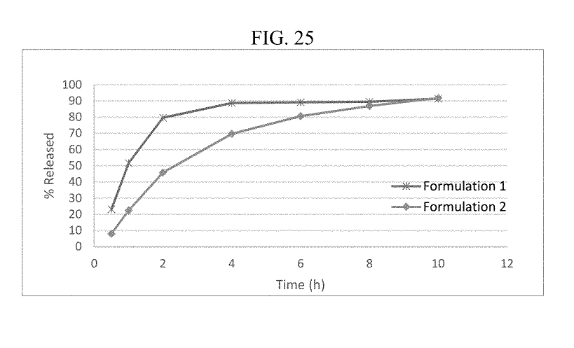

The capsule samples were analyzed by on-line UV detector. The drug release profiles from the capsules are presented in FIG. 25 and numerically as follows:

| | Time (hr) | | | | | | |
|---|---|---|---|---|---|---|---|
| | 0.5 | 1 | 2 | 4 | 6 | 8 | 10 |
| Formulation 1 | 23 | 52 | 80 | 89 | 89 | 89 | 91 |
| Formulation 2 | 8 | 22 | 46 | 70 | 81 | 87 | 92 |

### Example 19b

Extended-release capsules may be prepared as described in Example 19a by replacing the metoprolol succinate with other similarly water soluble drugs such as Tapentadol HCl, Oxycodone, HCl, Oxymorphone HCl, Hydrocodone Bitartrate, and Morphine Sulfate and obtain similar results. It is believed that the produced tablets will exhibit abuse deterrent properties.

It is envisioned that any feature or element that is positively identified in this description may also be specifically excluded as a feature or element of an embodiment of the present invention as defined in the claims.

The invention described herein may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A solid pharmaceutical dosage form comprising a plurality of drug-loaded particles wherein the drug-loaded particles comprise a core comprising a hydrophilic or gelling polymer and a drug-load applied to the core wherein the drug-load comprises a drug and a binder.

2. The solid pharmaceutical dosage form of claim 1 wherein the hydrophilic or gelling polymer is selected from the group consisting of polyethylene oxide, hydroxypropyl cellulose, hydroxypropyl methylcellulose, and a combination thereof.

3. The solid pharmaceutical dosage form of claim 1 wherein the binder is a hydrophobic or water-insoluble polymer is a water-insoluble film forming polymer.

4. The solid pharmaceutical dosage form of claim 3 wherein the hydrophobic or water-insoluble polymer is an ethyl acrylate and methyl methacrylate copolymer.

5. The solid pharmaceutical dosage form of claim 1 wherein the binder is a hydrophilic or water soluble polymer.

6. The solid pharmaceutical dosage form of claim 5 wherein the binder is hydroxypropyl methylcellulose, hydroxypropyl cellulose, hydroxyethyl cellulose, povidone, polyvinyl alcohol, copovidone, a pH dependent polymer or a combination of the foregoing.

7. The solid pharmaceutical dosage form of claim 1 further comprising an extra-granular component that comprises one or more excipients selected from the group consisting of hydrophilic or gelling polymers, fillers, binders, antioxidants, stabilizer flavoring agents, lubricants, glidants, or combinations thereof.

8. The dosage form of claim 7 wherein the extra-granular component comprises a hydrophilic polymer, an antioxidant, a glidant, and a lubricant.

9. The dosage form of claim 1 wherein the dosage form is a capsule or tablet.

10. The dosage form of claim 1 wherein the hydrophilic or gelling polymer is polyethylene oxide.

11. The dosage form of claim 10 wherein the polyethylene oxide comprises a polyethylene oxide having an average molecular weight less than 1,000,000 Daltons and a polyethylene oxide having an average molecular weight of greater than 1,000,000 Daltons.

12. The dosage form of claim 1 wherein the drug is a BCS class I drug.

13. The dosage form of claim 1 wherein the drug is a water soluble drug.

14. The dosage form of claim 1 wherein the drug is an opioid.

15. A process for preparing a solid dosage form comprising steps of:
(i) preparing a drug-loading solution, suspension, or dispersion comprising a drug, a binder, a solvent comprising water and optionally at least one additional pharmaceutical excipient;
(ii) spraying the drug-loading solution, suspension, or dispersion onto particles of a first hydrophilic polymer to form drug-loaded particles wherein the particles of the first hydrophilic polymer have an average particle size of about 25 μm to 800 μm and the particles of the first hydrophilic polymer are selected from the group consisting of: (a) alkyl substituted celluloses having a viscosity within the range of about 100 to about 11,000 centipoise as a 2% aqueous solution at 20° C., (b) polyethylene oxide having an approximate molecular weight of 100,000 Daltons to about 10,000,000 Daltons or (c) a combination of (a) and (b);
(iii) drying the drug-loaded particles;
(iv) optionally blending the drug-loaded particles with an extra-granular composition to form a final blend; and
(v) forming the drug-loaded particles into a dosage form.

16. The process of claim 15 further comprising the step of compressing the final blend into a tablet.

17. The process of claim 15 further comprising the step of filling the drug-loaded particles or the final blend into a capsule shell.

18. The process of claim 15 wherein the drug is a BCS class I drug.

19. The process of claim 15 wherein the drug is a water soluble drug.

20. The process of claim 15 wherein the drug is an opioid drug.

21. The process of claim 15 wherein the alkyl substituted cellulose is selected from the group consisting of hydroxypropyl cellulose, hydroxypropyl methylcellulose, and a combination thereof.

22. The process of claim 15 wherein the binder is a hydrophobic or water-insoluble polymer.

23. The process of claim 22 wherein the hydrophobic or water-insoluble polymer is an ethyl acrylate and methyl methacrylate copolymer.

24. The process of claim 15 wherein the binder is a hydrophilic or water soluble polymer.

25. The process of claim 24 wherein the binder is hydroxypropyl methylcellulose that exhibits a viscosity of less than 75 cps when a 2% aqueous solution is prepared, hydroxypropyl cellulose that exhibits a viscosity of less than 75 cps when a 2% aqueous solution is prepared, hydroxyethyl cellulose, povidone, polyvinyl alcohol, copovidone, a pH dependent polymer or a combination of the foregoing.
